(19) United States
(12) Patent Application Publication
(10) Pub. No.: US 2021/0025895 A1
(43) Pub. Date: Jan. 28, 2021

(54) CANCER SERUM BIOMARKERS AND METHODS OF USE THEREOF

(71) Applicant: X4 Pharmaceuticals, Inc., Cambridge, MA (US)

(72) Inventors: Yan Wang, Cambridge, MA (US); Simon Paul Fricker, North Grafton, MA (US); Kam Marie Sprott, Needham, MA (US)

(21) Appl. No.: 17/045,697

(22) PCT Filed: Apr. 12, 2019
(86) PCT No.: PCT/US2019/027169

§ 371 Date: Oct. 6, 2020

Related U.S. Application Data
(60) Provisional application No. 62/657,370, filed on Apr. 13, 2018.
(60) Provisional application No. 62/734,133, filed on Sep. 20, 2018.
(60) Provisional application No. 62/756,496, filed on Nov. 6, 2018.

(51) Int. Cl. G01N 33/574; G01N 33/50

(52) U.S. Cl. CPC G01N 33/57488; G01N 33/5088; G01N 2333/521; G01N 2333/52; G01N 2333/57; G01N 33/57438

(57) ABSTRACT

The present invention relates, in part, to certain serum biomarkers and use thereof in methods for treating cancer, such as in evaluating and/or in additional methods such as predicting patient responses to treatment with a CXCR4 inhibitor optionally in combination with a immunotherapeutic agent, in patients with a cancer such as melanoma, including resectable and unresectable melanoma.

### CROSS-REFERENCE TO RELATED APPLICATIONS

This application claims the benefit of U.S. Provisional Application No. 62/657,370, filed on Apr. 13, 2018; 62/734,133, filed on Sep. 20, 2018; and 62/756,496, filed on Nov. 6, 2018; the entirety of each of which is hereby incorporated by reference.

### FIELD OF THE INVENTION

The present invention relates generally to treatment of cancer using a CXCR4 inhibitor, alone or in combination with an immunotherapeutic agent. More specifically, the present invention relates, in part, to certain serum biomarkers and their use in methods for treating cancer, for example, in evaluating and/or predicting patient responses to treatment in patients.

### BACKGROUND OF THE INVENTION

The American Cancer Society's estimates for melanoma in the United States for 2017 are: about 87,110 new melanomas will be diagnosed (about 52,170 in men and 34,940 in women). About 9,730 people are expected to die of melanoma. The rates of melanoma have been rising for the last 30 years. When discovered early, melanoma is highly curable with 10-year overall survival rates approaching 95% for stage I melanoma and 45-77% for stage II melanoma after complete surgical resection of the primary melanoma. However, surgical treatment may not be feasible for all patients with advanced melanoma. Patients with unresectable or metastatic disease receive systemic treatment, including immunotherapy (e.g., checkpoint inhibitors (CPI) such as anti-PD-1 and anti-CTLA-4 antibodies) and targeted therapy (e.g., BRAF and/or MEK inhibitors for patients with known genetic mutations). Both checkpoint inhibitor immunotherapy and targeted therapy prolong progression-free survival and overall survival.

Moreover, 30% of patients who have undergone complete resection of their primary melanoma will develop local, in-transit and/or nodal recurrence of their disease. In addition, 10% of melanoma patients present with nodal metastases. Among these stage III patients, complete surgical removal is the main treatment for those with resectable disease; however, the risk of recurrence after surgery is very high. Adjuvant therapies with immunomodulating drugs such as high dose interferon-α and the anti-CTLA-4 antibody ipilimumab have shown to improve the recurrence-free survival in patients with resectable stage III melanoma. The impact of these adjuvant treatments on overall survival is not established.

Renal cell carcinoma is the seventh most common cancer in men and the ninth most common cancer in women in the United States, with an estimated 65,000 new cases and 13,500 deaths expected in 2015. While stage I, II and III are frequently treated by partial or radical nephrectomy, up to 30% of patients with localized tumors experience relapse. Cytoreductive nephrectomy, followed by systemic therapy is generally recommended in patients with stage IV renal cell carcinoma with a surgically resectable primary tumor. Systemic therapy is then recommended for patients with residual metastatic disease. Chittoria and Rini (2013) Renal Cell Carcinoma; www.clevelandclinicmeded.com/medicalpubs/diseasemanagement/nephrology/renal-cell-carcinoma/.

The benefit of neoadjuvant chemo- and immunotherapy has been demonstrated in several operable cancers. However, tumor development of resistance over time, e.g., via angiogenic escape, is frequently observed and limits the effectiveness of these therapies.

Investigation of CXCR4 inhibitors for use in treating a number of cancers is also warranted. CXCR4 was initially discovered for its involvement in HIV entry and leukocyte trafficking. It is also overexpressed in more than 23 human cancers. CXCR4 is frequently expressed on melanoma cells, particularly the CD133⁺ population that is considered to represent melanoma stem cells; in vitro experiments and murine models have demonstrated that CXCL12, the ligand for CXCR4, is chemotactic for such cells. These data underscore the significant, unmet need for study of CXCR4 inhibitors to treat cellular proliferative disorders that result from overexpression or aberrant expression of CXCR4.

### BRIEF DESCRIPTION OF THE DRAWINGS

FIG. 1 shows the dosage schedule for a nine (9) week study of X4P-001 monotherapy and in combination with pembrolizumab.

FIG. 2 shows the dosage schedule for a study of X4P-001 in combination with nivolumab in a renal cell carcinoma clinical trial.

FIG. 3 shows the target lesion response over time in a renal cell carcinoma clinical trial.

FIG. 4 shows the duration of prior nivolumab monotherapy and combination treatment and patient responses in a renal cell carcinoma clinical trial. Four patients with progressive disease on prior nivolumab monotherapy had a best response of stable disease (SD) with X4P-001+nivolumab. Among 5 patients with stable disease on prior nivolumab monotherapy, 1 had a partial response (PR) with X4P-001+nivolumab.

FIG. 5 shows an assessment of tumor responses by CT scans for a patient receiving X4P-001+nivolumab combination therapy that had a partial response in a renal cell carcinoma clinical trial. Top row: Target lesion in the lung. Bottom row: lymph node target lesion. Scans were taken every 8 weeks and target lesion size was determined per RECIST v1.1 criteria.

FIG. 6 shows measured increases in CXCL9 (MIG) levels in patients treated with X4P-001+nivolumab in a renal cell carcinoma clinical trial. Higher CXCL9 levels were found in a patient with a partial response (PR) and in those receiving combination therapy for >10 cycles.

FIG. 7 shows the changes observed in serum CXCL9 levels in response to X4P-001 monotherapy and combination therapy with X4P-001+pembrolizumab in a melanoma clinical trial.

FIG. 8 shows the changes observed in serum CXCL10 levels in response to X4P-001 monotherapy and combination therapy with X4P-001+pembrolizumab in a melanoma clinical trial.

### DETAILED DESCRIPTION OF CERTAIN EMBODIMENTS OF THE INVENTION

### General Description of Certain Embodiments of the Invention

Diagnosis, prognosis, and treatment of cancer is greatly aided by the identification of intratumoral expression patterns for sets of genes, changes in levels of immune-related cells in the tumor microenvironment, cytokine expression levels, or other changes in the tumor microenvironment, referred to herein generally as “biomarkers” or more specifically in relation to gene expression patterns as “gene signatures,” “gene expression biomarkers,” or “molecular signatures,” which are characteristic of particular types or subtypes of cancer, and which are associated with clinical outcomes. Such biomarkers may be associated with positive or negative clinical outcomes (e.g., increased or decreased likelihood of successful treatment, which may include increased quality of life and/or increased time of survival). If such an association is predictive of a clinical response, the biomarker is advantageously used in methods of selecting or stratifying patients as more (or less, as the case may be) likely to benefit from a treatment regimen, such as one of those disclosed herein. Tumor samples with biomarkers that are predictive of a positive response to treatment are referred to herein as “biomarker positive” or “biomarker high.” Conversely, tumor samples with biomarker profiles that are not predictive of a positive response are referred to herein as “biomarker negative” or “biomarker low.” Alternative terms can be used depending upon the biomarker, but a higher amount, or “biomarker high” usually can be described using alternative terminology, such as “biomarker positive” or “biomarker+” while a lower amount of a biomarker or “biomarker low” usually can be described using alternative terminology, such as “biomarker negative” or “biomarker-.”

In some embodiments, a biomarker used in the present invention is a biomarker panel, such as a cytokine panel. Such a “panel,” as used herein, refers to a group of specific biomarkers, e.g., cytokines, that respond to a particular stimulus (e.g., treatment of the patient with a CXCR4 inhibitor with an immunotherapeutic agent), in a way that tends to predict the likelihood of a particular clinical outcome. Individual biomarkers, e.g., cytokines, in a panel need not each respond in the same way. Some may be up-regulated and some may be down-regulated; accordingly, the overall response of the panel is generally the most useful in predicting the likelihood of a clinical response.

In some embodiments, a biomarker used in the present invention is a cytokine signature. Similar to a panel, a “signature” as used herein refers a group of biomarkers such as cytokines that respond to a stimulus to provide a fingerprint (distinctive pattern) of biomarker response to treatment.

Furthermore, while tumor derived biomarkers are an important tool in improving the diagnosis, prognosis, and treatment of cancers, the invasiveness of collecting tumor samples may increase the risk of metastasis (Shyamala, K., Girish, H. C., Murgod, S. J. Int. Prev. Comm. Dent. 4(1): 5-11 (2014)). Both the surgical removal of tumor tissue (biopsy) and the aspiration of tumor cells (fine needle aspiration cytology; FNAC) have the potential to drag tumor cells into neighboring tissues and/or expose abnormal cells to the lymphatic and/or circulatory systems. Additionally, the reduced invasiveness of collecting serum samples for biomarker analysis relative to biopsy or FNAC allows for more continuous monitoring of patient response to treatment. Consequently, minimally invasive diagnostic tools and methods that avoid disrupting tumor integrity or causing tumor inflammation, such as “serum biomarkers,” present opportunities to improve patient care while mitigating risks associated with current treatment regimens. Serum biomarkers include biomarkers that may be obtained by a bodily fluid sample obtained remote from a tumor (e.g., venous blood and lymph fluid). Examples of serum biomarkers include, for example, circulating cytokines and growth factors (e.g., interleukin IL-6, IL-10, and INF-γ), as well as phenotypic and genotypic markers in circulating cells (e.g., CD4, CD8, FoxP3, CD-127, and PD-1).

In some embodiments, the biomarker is selected from CXCL9 or CXCL10. In some embodiments, an increase in CXCL9 or CXCL10 is observed. In some embodiments, a decrease in CXCL9 or CXCL10 is observed.

In some embodiments, the biomarker is a cytokine panel.

In some embodiments, the cytokine panel comprises one or more biomarkers selected from ANG-1, ENA-78, Latency-associated peptide of transforming growth factor beta 1, MCP-1, and PDGF-BB. In some embodiments, the expression level of one or more of the above biomarkers is decreased after administration of a CXCR4 inhibitor.

In some embodiments, one, two, three, four, or five of the above biomarkers is decreased after administration of a CXCR4 inhibitor.

In some embodiments, the cytokine panel comprises one or more biomarkers selected from 6CKine, Decorin, IL-2, MIP-3 beta, MIG (CXCL9), and MPIF-1P. In some embodiments, the expression level of one or more of the above biomarkers is increased after administration of a CXCR4 inhibitor.

In some embodiments, one, two, three, four, five, or six of the above biomarkers are increased after administration of a CXCR4 inhibitor.

In some embodiments, the increase or decrease in the level of a serum biomarker in a patient is a measurable increase or decrease that correlates with an increased (or decreased, as the case may be) likelihood of therapeutic benefit for the patient, or for a group of patients, or a patient or group of patients yet to be selected. In some embodiments, the increase or decrease is a statistically significant increase or decrease. The term “statistical significance” is well-known in the art and may be determined using methods known in the art, such as those described herein. In some embodiments, statistical significance means, e.g., p<0.1, p<0.05, p<0.04, p<0.03, p<0.02, or p<0.01 relative to baseline.

In some embodiments, the increase or decrease in the level of a serum biomarker is observed after the patient has completed one cycle of treatment. In some embodiments, the increase or decrease is observed after two or more cycles of treatment, such as three, four, five, six, seven, eight, nine, or 10 or more cycles. The term “cycle of treatment” is well-known in the art and refers to a physician-defined treatment regimen followed by a patient for a period of time such as 1, 2, 3, or 4 weeks, optionally followed by a period of, e.g., 1, 2, 3, or 4 weeks of patient recovery and/or disease progression monitoring, during which, in some cases, a lower dose of therapeutic agent (or no therapeutic agent at all) is administered. In some embodiments, a cycle of treatment refers to administering a CXCR4 inhibitor, such as X4P-001 or a pharmaceutically acceptable salt thereof, either as a monotherapy, or in combination with a checkpoint inhibitor, such as nivolumab or pembrolizumab, in cycles, such as on a 2 week, 4 week, 6 week or 8 week cycle. In certain embodiments, the cycle is 4 weeks long. In some embodiments, X4P-001 or a pharmaceutically acceptable salt thereof is administered at a determined dose from 200 mg to 1200 mg daily. In some embodiments, the administration is orally either once daily or twice daily in divided doses. In some embodiments, the dose is about 400 mg per day. In some embodiments, oral X4P-001 is administered to patients at 400 mg once per day (QD) in combination with about 240 mg nivolumab therapy by IV infusion approximately every 2 weeks.

It has been surprisingly found that levels of serum cytokines are useful as biomarkers in a method described herein, such as a method of treating or diagnosing a cancer such as metastatic melanoma or renal cell carcinoma (RCC).

In one aspect, the present invention provides a method of identifying a patient with a cancer who will benefit from treatment with a CXCR4 inhibitor optionally in combination with an immunotherapeutic agent, comprising:
(a) obtaining a first serum sample prior to administration of the CXCR4 inhibitor to the patient;
(b) measuring a level in the first serum sample of one or more biomarkers selected from a cytokine panel, a cytokine signature, a ratio of one or more cytokine ratios, or a cytokine score;
(c) administering to the patient an effective amount of the CXCR4 inhibitor and optionally the immunotherapeutic agent;
(d) obtaining a second serum sample after administration of the CXCR4 inhibitor and optionally the immunotherapeutic agent to the patient; and
(e) measuring a level in the second serum sample of one or more biomarkers selected from a cytokine panel, a cytokine signature, a ratio of one or more cytokine ratios, or a cytokine score;
   wherein the cancer response to step (c) is predictive of the likelihood of successful treatment of the cancer based on a greater or lesser response of the cancer compared with one or more similar patients and as evaluated using one or more of the biomarkers.

In some embodiments, the biomarker is selected from CXCL9 or CXCL10.

In some embodiments, the biomarker is a cytokine panel. In some embodiments, the cytokine panel comprises one or more biomarkers selected from ANG-1, ENA-78, Latency-associated peptide of transforming growth factor beta 1, MCP-1, and PDGF-BB. In some embodiments, the expression level of one or more of the above biomarkers is decreased after administration of a CXCR4 inhibitor.

In some embodiments, the cytokine panel comprises one or more biomarkers selected from 6CKine, Decorin, IL-2, MIP-3 beta, MIG (CXCL9), and MPIF-1P. In some embodiments, the expression level of one or more of the above biomarkers is increased after administration of a CXCR4 inhibitor.

In some embodiments, the CXCR4 inhibitor is administered in combination with an immunotherapeutic agent. In some embodiments, the CXCR4 inhibitor is X4P-001 or a pharmaceutically acceptable salt thereof. X4P-001 has the structure depicted below:

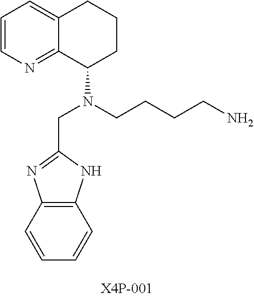 [SMILES: NCCCCN(Cc1nc2ccccc2[nH]1)[C@H]1CCCc2cccnc21]

X4P-001 and the synthesis thereof is described in detail in U.S. Pat. No. 7,354,934, which is hereby incorporated by reference.

In some embodiments, the immunotherapeutic agent is a checkpoint inhibitor. In some embodiments, the checkpoint inhibitor is a PD-1 antagonist. In some embodiments, the PD-1 antagonist is selected from nivolumab, pembrolizumab, a pembrolizumab biosimilar, or a pembrolizumab variant. In some embodiments, the checkpoint inhibitor is pembrolizumab.

In some embodiments, the cancer is a cancerous tumor. In some embodiments, the cancerous tumor is a solid tumor. In some embodiments, the solid tumor is melanoma. In some embodiments, the melanoma is malignant melanoma, advanced melanoma, metastatic melanoma, or Stage I, II, III, or IV melanoma. In some embodiments, the melanoma is resectable. In some embodiments, the melanoma is unresectable. In some embodiments, the melanoma is unresectable advanced or unresectable metastatic melanoma.

In some embodiments, the patient has not previously undergone treatment with an immune checkpoint inhibitor such as anti-CTLA-4, PD-1, or PD-L1, or previously undergone oncolytic virus therapy.

In some embodiments, the above method is useful in the identification of a patient who will benefit from treatment with a CXCR4 inhibitor optionally in combination with an immunotherapeutic agent. Such a patient is characterized in that the level of one or more biomarkers selected from a cytokine panel, a cytokine signature, a ratio of one or more cytokine ratios, or a cytokine score is altered (i.e., higher or lower) in the second serum sample. This is because such a patient is considered likely to benefit from continued treatment with the CXCR4 inhibitor and, optionally, the immunotherapeutic agent.

In another aspect, the present invention provides a method of identifying a patient with a cancer who is likely to benefit from treatment with a CXCR4 inhibitor optionally in combination with an immunotherapeutic agent, comprising:
(a) obtaining a first serum sample prior to administration of the CXCR4 inhibitor to the patient;
(b) measuring a level in the first serum sample of one or more biomarkers selected from a cytokine panel, a cytokine signature, a ratio of one or more cytokine ratios, or a cytokine score;
(c) administering to the patient an effective amount of the CXCR4 inhibitor and optionally the immunotherapeutic agent;
(d) obtaining a second serum sample after administration of the CXCR4 inhibitor and optionally the immunotherapeutic agent to the patient; and
(e) measuring a level in the second serum sample of one or more biomarkers selected from a cytokine panel, a cytokine signature, a ratio of one or more cytokine ratios, or a cytokine score;
   wherein the cancer response to step (c) is predictive of the likelihood of successful treatment of the cancer based on a greater or lesser response of the cancer compared with one or more similar patients and as evaluated using one or more of the biomarkers.

In some embodiments, the biomarker is selected from CXCL9 or CXCL10.

In some embodiments, the biomarker is a cytokine panel. In some embodiments, the cytokine panel comprises one or more biomarkers selected from ANG-1, ENA-78, Latency-associated peptide of transforming growth factor beta 1, MCP-1, and PDGF-BB. In some embodiments, the expression level of one or more of the above biomarkers is decreased after administration of a CXCR4 inhibitor.

In some embodiments, the cytokine panel comprises one or more biomarkers selected from 6CKine, Decorin, IL-2, MIP-3 beta, MIG (CXCL9), and MPIF-1P. In some embodiments, the expression level of one or more of the above biomarkers is increased after administration of a CXCR4 inhibitor.

In some embodiments, the patient's biomarker levels correlate with one or more similar patients. In some embodiments, the correlated biomarkers are indicative of an increased or decreased likelihood of successful treatment improved likelihood of successful treatment. In some embodiments, the correlated biomarkers are indicative of an increased likelihood of successful treatment. In some embodiments, the correlated biomarkers are indicative of an increased likelihood of successful treatment, but the cancer has not yet responded to treatment.

In another aspect, the present invention provides a method of treating a cancer with a CXCR4 inhibitor optionally in combination with an immunotherapeutic agent, comprising:
(a) obtaining a first serum sample prior to administration of the CXCR4 inhibitor to the patient;
(b) measuring a level in the first serum sample of one or more biomarkers selected from a cytokine panel, a cytokine signature, a ratio of one or more cytokine ratios, or a cytokine score;
(c) administering to the patient an effective amount of a CXCR4 inhibitor and optionally the immunotherapeutic agent;
(d) obtaining a second serum sample after administration of the CXCR4 inhibitor and optionally the immunotherapeutic agent to the patient; and
(e) measuring a level in the second serum sample of one or more biomarkers selected from a cytokine panel, a cytokine signature, a ratio of one or more cytokine ratios, or a cytokine score; wherein:
   when the level of one or more biomarkers selected from a cytokine panel, a cytokine signature, a ratio of one or more cytokine ratios, or a cytokine score is higher in the second venous blood sample than in the first serum sample, then the patient is administered one or more additional doses of the CXCR4 inhibitor and optionally the immunotherapeutic agent.

In some embodiments, the biomarker is selected from CXCL9 or CXCL10.

In some embodiments, the biomarker is a cytokine panel. In some embodiments, the cytokine panel comprises one or more biomarkers selected from ANG-1, ENA-78, Latency-associated peptide of transforming growth factor beta 1, MCP-1, and PDGF-BB. In some embodiments, the expression level of one or more of the above biomarkers is decreased after administration of a CXCR4 inhibitor.

In some embodiments, the cytokine panel comprises one or more biomarkers selected from 6CKine, Decorin, IL-2, MIP-3 beta, MIG (CXCL9), and MPIF-1P. In some embodiments, the expression level of one or more of the above biomarkers is increased after administration of a CXCR4 inhibitor.

In another aspect, the present invention provides a method of evaluating a cancer patient's response to a CXCR4 inhibitor optionally in combination with an immunotherapeutic agent, comprising the steps of:
(a) obtaining a first serum sample prior to administration of the CXCR4 inhibitor to the patient;
(b) measuring a level in the first serum sample of one or more biomarkers selected from a cytokine panel, a cytokine signature, a ratio of one or more cytokine ratios, or a cytokine score;
(c) administering to the patient an effective amount of a CXCR4 inhibitor and optionally the immunotherapeutic agent;
(d) obtaining a second serum sample after administration of the CXCR4 inhibitor and optionally the immunotherapeutic agent to the patient; and
(e) measuring a level in the second serum sample of one or more biomarkers selected from a cytokine panel, a cytokine signature, a ratio of one or more cytokine ratios, or a cytokine score;
   wherein the cancer response to step (c) is evaluated to split, classify, or stratify the patient into one of two or more groups based on a greater or lesser response of the cancer compared with one or more similar patients.

In another aspect, the present invention provides a method of predicting a cancer patient's response to a CXCR4 inhibitor optionally in combination with an immunotherapeutic agent, comprising the steps of:
(a) obtaining a first serum sample prior to administration of the CXCR4 inhibitor to the patient;
(b) measuring a level in the first serum sample of one or more biomarkers selected from a cytokine panel, a cytokine signature, a ratio of one or more cytokine ratios, or a cytokine score;
(c) administering to the patient an effective amount of the CXCR4 inhibitor and optionally the immunotherapeutic agent;
(d) obtaining a second serum sample after administration of the CXCR4 inhibitor to the patient; and
(e) measuring a level in the second serum sample of one or more biomarkers selected from a cytokine panel, a cytokine signature, a ratio of one or more cytokine ratios, or a cytokine score;
   wherein the cancer response to step (c) is predictive of the likelihood of successful treatment of the cancer based on a greater or lesser response of the cancer compared with one or more similar patients and as evaluated using one or more of the biomarkers.

In another aspect, the present invention provides a method of predicting a treatment response of a cancer in a patient to a CXCR4 inhibitor optionally in combination with an immunotherapeutic agent, comprising the steps of:
(a) obtaining a serum sample prior to administration of the CXCR4 inhibitor to the patient;
(b) measuring a level in the first serum sample of one or more biomarkers selected from a cytokine panel, a cytokine signature, a ratio of one or more cytokine ratios, or a cytokine score;
(c) treating the serum sample or a reference sample;
(e) measuring a level in the treated serum sample of one or more biomarkers selected from a cytokine panel, a cytokine signature, a ratio of one or more cytokine ratios, or a cytokine score; and
(f) comparing one of more biomarkers in the pre-treatment serum sample with one or more biomarkers in the treated serum sample or treated reference sample; and
(g) optionally, proceeding with administration of the CXCR4 inhibitor to the patient, optionally in combination with the immunotherapeutic agent, if such administration is predicted to have an equivalent or higher likelihood of success relative to an alternative method of treating the cancer;
   wherein the biomarker change in response to step (c) is predictive of the likelihood of successful treatment of the cancer based on a greater or lesser biomarker change compared with one or more similar patients and as evaluated using one or more of the biomarkers.

In some embodiments, the reference sample is from another patient, such as a patient with a similar cancer; or the reference sample may be a culture or other in vitro sample of a similar cancer.

In another aspect, the present invention provides a method of predicting a treatment response of a cancer in a patient to an immunotherapeutic agent in combination with a CXCR4 inhibitor, comprising the steps of:
(a) obtaining a first serum sample from a patent prior to the administration of the CXCR4 inhibitor to the patient;
(b) measuring a level in the first serum sample of one or more biomarkers selected from a cytokine panel, cytokine signature, a ratio of one or more cytokine ratios, or a cytokine score;
(c) administering to the patient an effective amount of a CXCR4 inhibitor and optionally an immunotherapeutic agent;
(d) obtaining a second serum sample after administration of the CXCR4 inhibitor to the patient;
(e) measuring a level in the second serum sample of one or more biomarkers selected from a cytokine panel, cytokine signature, a ratio of one or more cytokine ratios, or a cytokine score;
   wherein the tumor response to step (c) is predictive of the likelihood of successful treatment of the tumor with an immunotherapeutic agent after treatment with a CXCR4 inhibitor, based on a greater of lesser response of the tumor compared with one or more similar patients and as evaluated using one or more biomarkers.

In some embodiments, the immunotherapeutic agent is a checkpoint inhibitor. In some embodiments, the immune checkpoint inhibitor is an anti-CTLA-4, PD-1, or PD-L1.

In some embodiments, the patient has not previously undergone treatment with an immune checkpoint inhibitor. In some embodiments, the patient has previously undergone treatment with an immune checkpoint inhibitor.

In some embodiments, the cancer is refractory to immune checkpoint inhibitors. In some embodiments, the cancer was initially responsive to treatment with an immune checkpoint inhibitor, but has become refractory to treatment with the immune checkpoint inhibitor.

In some embodiments, the biomarker is selected from CXCL9 or CXCL10.

In some embodiments, the biomarker is a cytokine panel. In some embodiments, the cytokine panel comprises one or more biomarkers selected from ANG-1, ENA-78, Latency-associated peptide of transforming growth factor beta 1, MCP-1, and PDGF-BB. In some embodiments, the expression level of one or more of the above biomarkers is decreases after administration of a CXCR4 inhibitor.

In some embodiment, the cytokine panel comprises one or more biomarkers selected from 6CKine, Decorin, IL-1, MIP-3 beta, MIG (CXCL9, and MPIF-1P. In some embodiments, the expression level of one or more of the above biomarkers is increased after administration of a CXCR4 inhibitor.

In another aspect, the present invention provides a method of monitoring a cancer patient's response to a CXCR4 inhibitor optionally in combination with an immunotherapeutic agent, comprising the steps of:
(a) obtaining a first serum sample prior to administration of the CXCR4 inhibitor to the patient;
(b) measuring a level in the first serum sample of one or more biomarkers selected from a cytokine panel, a cytokine signature, a ratio of one or more cytokine ratios, or a cytokine score;
(c) administering to the patient an effective amount of the CXCR4 inhibitor and optionally the immunotherapeutic agent;
(d) obtaining a subsequent serum samples after administration of the CXCR4 inhibitor to the patient; and
(e) measuring a level in the subsequent serum samples of one or more biomarkers selected from a cytokine panel, a cytokine signature, a ratio of one or more cytokine ratios, or a cytokine score;
   wherein the levels of one of more biomarkers can in the pre-treatment serum sample and subsequent serum samples can be compared and a greater or lesser change in one or more of the biomarkers is indicative of a positive response.

In some embodiments, the patient's response to a CXCR4 inhibitor optionally in combination with an immunotherapeutic agent is measured once per week or every two weeks. In some embodiments, the patient's response is measured once a month. In some embodiments, the patient's response is measured bimonthly. In some embodiments, the patient's response is measured quarterly (once every three months). In some embodiments, the patient's response is measured annually.

In some embodiments, the patient's response to a CXCR4 inhibitor optionally in combination with an immunotherapeutic agent is monitored while undergoing treatment. In some embodiments, the patient's response is monitored after treatment is concluded.

In some embodiments, the biomarker is selected from CXCL9 or CXCL10.

In some embodiments, the biomarker is a cytokine panel. In some embodiments, the cytokine panel comprises one or more biomarkers selected from ANG-1, ENA-78, Latency-associated peptide of transforming growth factor beta 1, MCP-1, and PDGF-BB. In some embodiments, the expression level of one or more of the above biomarkers is decreased after administration of a CXCR4 inhibitor.

In some embodiments, the cytokine panel comprises one or more biomarkers selected from 6CKine, Decorin, IL-2, MIP-3 beta, MIG (CXCL9), and MPIF-1P. In some embodiments, the expression level of one or more of the above biomarkers is increased after administration of a CXCR4 inhibitor.

In another aspect, the present invention provides a method of deriving a biomarker signature that is predictive of an anti-cancer response to treatment with a CXCR4 inhibitor optionally in combination with a PD-1 antagonist for a cancer, comprising:
(a) obtaining a pre-treatment serum sample from each patient in a patient cohort diagnosed with the cancer type;
(b) obtaining, for each patient in the cohort, an anti-cancer response value following treatment with the CXCR4 inhibitor optionally in combination with the PD-1 antagonist;
(c) measuring the raw biomarker levels in each serum sample for each biomarker in a biomarker platform, wherein the biomarker platform comprises a clinical response biomarker set of a cytokine score;
(d) normalizing, for each serum sample, each of the measured raw biomarker levels for the clinical response biomarkers using the measured biomarker levels of a set of normalization biomarkers; and
(e) comparing the biomarker levels for all of the serum samples and the anti-cancer response values for all of the patients in the cohort to select a cutoff for the biomarker signature score that divides the patient cohort to meet a target biomarker clinical utility criterion.

In some embodiments, the biomarker is selected from CXCL9 or CXCL10.

In some embodiments, the biomarker is a cytokine panel. In some embodiments, the cytokine panel comprises one or more biomarkers selected from ANG-1, ENA-78, Latency-associated peptide of transforming growth factor beta 1, MCP-1, and PDGF-BB. In some embodiments, the expression level of one or more of the above biomarkers is decreased after administration of a CXCR4 inhibitor.

In some embodiments, the cytokine panel comprises one or more biomarkers selected from 6CKine, Decorin, IL-2, MIP-3 beta, MIG (CXCL9), and MPIF-1P. In some embodiments, the expression level of one or more of the above biomarkers is increased after administration of a CXCR4 inhibitor.

In some embodiments, the biomarker platform comprises a gene expression platform that comprises a clinical response gene set. In some embodiments, the method further comprises the steps of:
(f) weighting, for each serum sample and each biomarker, such as a gene in a gene signature of interest, the normalized biomarker (e.g., RNA biomarker) expression levels using a pre-defined multiplication coefficient for that gene;
(g) adding, for each patient, the weighted biomarker (e.g., RNA biomarker) expression levels to generate a biomarker signature score, e.g., a gene signature score, for each patient in the cohort.

In some embodiments, the biomarker is selected from CXCL9 or CXCL10.

In some embodiments, the biomarker is a cytokine panel. In some embodiments, the cytokine panel comprises one or more biomarkers selected from ANG-1, ENA-78, Latency-associated peptide of transforming growth factor beta 1, MCP-1, and PDGF-BB. In some embodiments, the expression level of one or more of the above biomarkers is decreased after administration of a CXCR4 inhibitor.

In some embodiments, the cytokine panel comprises one or more biomarkers selected from 6CKine, Decorin, IL-2, MIP-3 beta, MIG (CXCL9), and MPIF-1P. In some embodiments, the expression level of one or more of the above biomarkers is increased after administration of a CXCR4 inhibitor.

In another aspect, the present invention provides a method of testing a serum sample removed from a patient for the presence or absence of a biomarker signature of anti-cancer response of a cancer to a CXCR4 inhibitor optionally in combination with a PD-1 antagonist, comprising:
(a) measuring the raw biomarker level in the serum sample for each biomarker in a biomarker platform, wherein the biomarker platform comprises a clinical response biomarker set selected from a cytokine panel, a cytokine signature, a ratio of one or more cytokine ratios, or a cytokine score and a normalization biomarker set, and optionally wherein about 80%, or about 90%, of the clinical response biomarkers exhibit serum biomarker levels that are positively correlated with the anti-cancer response;
(b) normalizing the measured raw biomarker level for each clinical response biomarker in a pre-defined biomarker signature for the serum sample using the measured biomarker levels of the normalization biomarkers, wherein the pre-defined biomarker signature consists of at least 2 of the clinical response biomarkers;
(c) comparing the normalized biomarker levels and a set of reference biomarker levels for a cancer; and
(d) classifying the serum sample as biomarker high or biomarker low:
   wherein if the normalized biomarker levels are equal to or greater than the reference biomarker levels, then the serum sample is classified as biomarker high, and if the normalized biomarker levels are less than the reference biomarker levels, then the serum sample is classified as biomarker low.

In some embodiments, the biomarker is selected from CXCL9 or CXCL10.

In some embodiments, the biomarker is a cytokine panel. In some embodiments, the cytokine panel comprises one or more biomarkers selected from ANG-1, ENA-78, Latency-associated peptide of transforming growth factor beta 1, MCP-1, and PDGF-BB. In some embodiments, the expression level of one or more of the above biomarkers is decreased after administration of a CXCR4 inhibitor.

In some embodiments, the cytokine panel comprises one or more biomarkers selected from 6CKine, Decorin, IL-2, MIP-3 beta, MIG (CXCL9), and MPIF-1P. In some embodiments, the expression level of one or more of the above biomarkers is increased after administration of a CXCR4 inhibitor.

In some embodiments, after step (b) the method comprises the further steps of:

(i) weighting each normalized biomarker value using a pre-defined multiplication co-efficient;

(ii) adding the weighted biomarker levels to generate a weighted biomarker signature score.

In some embodiments, utilizes a normalization gene set comprising about 10 to about 12 housekeeping genes, or about 30-40 housekeeping genes.

In another aspect, the present invention provides a method of testing a serum sample removed from a patient for the presence or absence of a biomarker signature of anti-cancer response of the cancer to a CXCR4 inhibitor optionally in combination with a PD-1 antagonist, comprising:
(a) measuring the raw biomarker level in the serum sample for each biomarker in a biomarker platform, wherein the biomarker platform comprises a clinical response biomarker set selected from a cytokine panel, a cytokine signature, a ratio of one or more cytokine ratios, or a cytokine score and a normalization biomarker set, and optionally wherein about 80%, or about 90%, of the clinical response biomarkers exhibit intratumoral biomarker levels that are positively correlated with the anti-cancer response;
(b) normalizing the measured raw biomarker level for each clinical response biomarker in a pre-defined biomarker signature for the serum sample using the measured biomarker levels of the normalization biomarkers, wherein the pre-defined biomarker signature consists of at least 2 of the clinical response biomarkers;
(c) comparing the normalized biomarker levels and a set of reference biomarker levels for the serum sample; and
(d) classifying the serum sample as biomarker high or biomarker low;
   wherein if the normalized biomarker levels are equal to or greater than the reference biomarker levels, then the serum sample is classified as biomarker high, and if the normalized biomarker levels are less than the reference biomarker levels, then the serum sample is classified as biomarker low.

In another aspect, the present invention provides a system for testing a serum sample removed from a patient for the presence or absence of a biomarker signature of anti-cancer response of the cancer to a CXCR4 inhibitor optionally in combination with a PD-1 antagonist, comprising:
(i) a sample analyzer for measuring raw biomarker levels in a biomarker platform, wherein the biomarker platform consists of a set of clinical response biomarkers selected from a cytokine panel, a cytokine signature, a ratio of one or more cytokine ratios, or a cytokine score; and a set of normalization biomarkers; and
(ii) a computer program for receiving and analyzing the measured biomarker levels to:
   (a) normalize the measured raw biomarker level for each clinical response biomarker in a pre-defined biomarker signature for a cancer using the measured levels of the normalization biomarkers;
   (b) compare the generated biomarker level to a reference level for the biomarker signature and cancer; and
   (c) classify the serum sample as biomarker high or biomarker low, wherein if the generated score is equal to or greater than the reference score, then the serum sample is classified as biomarker high, and if the generated score is less than the reference score, then the serum sample is classified as biomarker low.

In another aspect, the present invention provides a system for testing a serum sample removed from a patient for the presence or absence of a biomarker signature of anti-cancer response of the cancer to a CXCR4 inhibitor optionally in combination with a PD-1 antagonist, comprising:
(i) a sample analyzer for measuring raw biomarker levels in a biomarker platform, wherein the biomarker platform consists of a set of clinical response biomarkers selected from a cytokine panel, a cytokine signature, a ratio of one or more cytokine ratios, or a cytokine score; and a set of normalization biomarkers; and
(ii) a computer program for receiving and analyzing the measured biomarker levels to:
   (a) normalize the measured raw biomarker level for each clinical response biomarker in a pre-defined biomarker signature for the cancer using the measured levels of the normalization biomarkers;
   (b) weight each normalized biomarker level using a pre-defined multiplication coefficient;
   (c) add the weighted biomarker levels to generate a biomarker signature score;
   (d) compare the generated score to a reference score for the biomarker signature and cancer; and
   (e) classify the serum sample as biomarker high or biomarker low, wherein if the generated score is equal to or greater than the reference score, then the serum sample is classified as biomarker high, and if the generated score is less than the reference score, then the serum sample is classified as biomarker low.

In some embodiments, the biomarker is selected from CXCL9 or CXCL10.

In some embodiments, the biomarker is a cytokine panel. In some embodiments, the cytokine panel comprises one or more biomarkers selected from ANG-1, ENA-78, Latency-associated peptide of transforming growth factor beta 1, MCP-1, and PDGF-BB. In some embodiments, the expression level of one or more of the above biomarkers is decreased after administration of a CXCR4 inhibitor.

In some embodiments, the cytokine panel comprises one or more biomarkers selected from 6CKine, Decorin, IL-2, MIP-3 beta, MIG (CXCL9), and MPIF-1P. In some embodiments, the expression level of one or more of the above biomarkers is increased after administration of a CXCR4 inhibitor.

In some embodiments, the biomarker comprises the RNA expression level of a gene described herein, such as a cytokine signature score. In some embodiments, the biomarker further comprises CD8A, CD8B, FoxP3, granzyme B, an IFN-γ signature gene, a CTL signature gene, an antigen presentation/processing signature gene, a tumor inflammation signature gene, or PD-L1 expression. In some embodiments, the biomarker further comprises levels of CD3 and/or Ki67, or CD4, CXCR4, CXCL12, arginase, FAPalpha, CD33 or CD11b. In some embodiments, the biomarker comprises levels of CD8⁺ T cells or CD8⁺ T cells/T_{reg} ratio or granzyme B levels. In some embodiments, such levels are measured by immunohistochemistry staining.

In another aspect, the present invention provides a kit for assaying a serum sample from a patient treated with a CXCR4 inhibitor optionally in combination with a PD-1 antagonist to obtain normalized RNA expression scores for a gene signature associated with the cancer, wherein the kit comprises:
(a) a set of hybridization probes capable of specifically binding to a transcript expressed by each of the genes; and
(b) a set of reagents designed to quantify the number of specific hybridization complexes formed with each hybridization probe. In some embodiments, the gene signature is selected from a cytokine signature score.

In another aspect, the present invention provides a method for treating a patient having a cancer, comprising the steps of: determining if a sample of serum is positive or negative for a gene signature biomarker; and administering to the patient a CXCR4 inhibitor optionally in combination with a PD-1 antagonist if the serum is positive for the biomarker and administering to the subject a cancer treatment that does not include a CXCR4 inhibitor or PD-1 antagonist if the serum is negative for the biomarker; wherein the gene signature biomarker is for a gene signature that comprises at least two of the clinical response genes selected from a cytokine signature score. In some embodiments, a multi-gene signature score, such as an IFN-γ, a CTL, an antigen presentation/processing, or a tumor inflammation signature score can be used as one “biomarker” in the same grouping as other individual gene biomarkers, to calculate a more predictive gene signature score.

In another aspect, the present invention provides a method of testing a serum sample removed from a patient to generate a signature score for a gene signature that is correlated with an anti-cancer response to a CXCR4 inhibitor, optionally in combination with a PD-1 antagonist, wherein the method comprises:
(a) measuring the raw RNA level in the serum sample for each gene in the gene signature and for each gene in a normalization gene set, wherein the gene signature comprises a cytokine signature score;
(b) normalizing the measured raw RNA level for each gene in the gene signature using the measured RNA levels of the normalization genes;
(c) multiplying each normalized RNA value by a calculated scoring weight to generate a weighted RNA expression value; and
(d) adding the weighted RNA expression values to generate the gene signature score.

In some embodiments, the method of identifying a patient with a cancer who will benefit from treatment further comprises one or more additional biomarkers selected from CD8⁺ T cells (or CD8⁺ T cells/T_{reg} ratio), CD8⁺Ki-67⁺ T cells, granzyme B, an IFN-γ signature score, CTL signature score, an antigen presentation/processing signature score, a tumor inflammation signature score, or PD-L1 expression, obtained from a collected tumor sample.

In some embodiments, the additional biomarker is a cytokine panel. In some embodiments, the cytokine panel comprises one or more biomarkers selected from ANG-1, ENA-78, Latency-associated peptide of transforming growth factor beta 1, MCP-1, and PDGF-BB. In some embodiments, the expression level of one or more of the above biomarkers is decreased after administration of a CXCR4 inhibitor. In some embodiments, the cytokine panel comprises one or more biomarkers selected from 6CKine, Decorin, IL-2, MIP-3 beta, MIG (CXCL9), and MPIF-1P. In some embodiments, the expression level of one or more of the above biomarkers is increased after administration of a CXCR4 inhibitor.

In some embodiments, the method of treating a cancer with a CXCR4 inhibitor further comprises one or more additional biomarkers selected from CD8⁺ T cells (or CD8⁺ T cells/T_{reg} ratio), CD8⁺Ki-67⁺ T cells, granzyme B, an IFN-γ signature score, CTL signature score, an antigen presentation/processing signature score, a tumor inflammation signature score, or PD-L1 expression, obtained from a collected tumor sample.

In some embodiments, the additional biomarker is a cytokine panel. In some embodiments, the cytokine panel comprises one or more biomarkers selected from ANG-1, ENA-78, Latency-associated peptide of transforming growth factor beta 1, MCP-1, and PDGF-BB. In some embodiments, the expression level of one or more of the above biomarkers is decreased after administration of a CXCR4 inhibitor. In some embodiments, the cytokine panel comprises one or more biomarkers selected from 6CKine, Decorin, IL-2, MIP-3 beta, MIG (CXCL9), and MPIF-1P. In some embodiments, the expression level of one or more of the above biomarkers is increased after administration of a CXCR4 inhibitor.

In some embodiments, the method of evaluating a patient response to a CXCR4 inhibitor optionally in combination with an immunotherapeutic agent further comprises one or more additional biomarkers selected from CD8⁺ T cells (or CD8⁺ T cells/T_{reg} ratio), CD8⁺Ki-67⁺ T cells, granzyme B, an IFN-γ signature score, CTL signature score, an antigen presentation/processing signature score, a tumor inflammation signature score, or PD-L1 expression, obtained from a collected tumor sample.

In some embodiments, the additional biomarker is a cytokine panel. In some embodiments, the cytokine panel comprises one or more biomarkers selected from ANG-1, ENA-78, Latency-associated peptide of transforming growth factor beta 1, MCP-1, and PDGF-BB. In some embodiments, the expression level of one or more of the above biomarkers is decreased after administration of a CXCR4 inhibitor. In some embodiments, the cytokine panel comprises one or more biomarkers selected from 6CKine, Decorin, IL-2, MIP-3 beta, MIG (CXCL9), and MPIF-1P. In some embodiments, the expression level of one or more of the above biomarkers is increased after administration of a CXCR4 inhibitor.

In some embodiments, the method of predicting a patient response to a CXCR4 inhibitor optionally in combination with an immunotherapeutic agent further comprises one or more additional biomarkers selected from CD8⁺ T cells (or CD8⁺ T cells/T_{reg} ratio), CD8⁺Ki-67⁺ T cells, granzyme B, an IFN-γ signature score, CTL signature score, an antigen presentation/processing signature score, a tumor inflammation signature score, or PD-L1 expression, obtained from a collected tumor sample.

In some embodiments, the additional biomarker is a cytokine panel. In some embodiments, the cytokine panel comprises one or more biomarkers selected from ANG-1, ENA-78, Latency-associated peptide of transforming growth factor beta 1, MCP-1, and PDGF-BB. In some embodiments, the expression level of one or more of the above biomarkers is decreased after administration of a CXCR4 inhibitor. In some embodiments, the cytokine panel comprises one or more biomarkers selected from 6CKine, Decorin, IL-2, MIP-3 beta, MIG (CXCL9), and MPIF-1P. In some embodiments, the expression level of one or more of the above biomarkers is increased after administration of a CXCR4 inhibitor.

In some embodiments, the method of deriving a biomarker signature that is predictive of an antitumor response to treatment with a CXCR4 inhibitor optionally in combination with a PD-1 antagonist for a tumor further comprises a clinical response biomarker set of CD8⁺ T cells or CD8⁺ T cells/T_{reg} ratio, CD8⁺Ki-67⁺ T cells, granzyme B, an IFN-γ signature score, CTL signature score, an antigen presentation/processing signature score, a tumor inflammation signature score, or PD-L1 expression, obtained from a collected tumor sample.

In some embodiments, the additional biomarker is a cytokine panel. In some embodiments, the cytokine panel comprises one or more biomarkers selected from ANG-1, ENA-78, Latency-associated peptide of transforming growth factor beta 1, MCP-1, and PDGF-BB. In some embodiments, the expression level of one or more of the above biomarkers is decreased after administration of a CXCR4 inhibitor. In some embodiments, the cytokine panel comprises one or more biomarkers selected from 6CKine, Decorin, IL-2, MIP-3 beta, MIG (CXCL9), and MPIF-1P. In some embodiments, the expression level of one or more of the above biomarkers is increased after administration of a CXCR4 inhibitor.

In some embodiments, the method of testing a serum sample removed from a patient for the presence or absence of a gene signature biomarker of anti-tumor response of the tumor to a CXCR4 inhibitor optionally in combination with a PD-1 antagonist further comprises a clinical response gene set selected from an IFN-γ signature, CTL signature, antigen presentation/processing signature, a tumor inflammation signature, CD8A, CD8B, granzyme B gene expression, or PD-L1 expression, obtained from a tumor sample removed from the patient.

In some embodiments, the additional biomarker is a cytokine panel. In some embodiments, the cytokine panel comprises one or more biomarkers selected from ANG-1, ENA-78, Latency-associated peptide of transforming growth factor beta 1, MCP-1, and PDGF-BB. In some embodiments, the expression level of one or more of the above biomarkers is decreased after administration of a CXCR4 inhibitor. In some embodiments, the cytokine panel comprises one or more biomarkers selected from 6CKine, Decorin, IL-2, MIP-3 beta, MIG (CXCL9), and MPIF-1P. In some embodiments, the expression level of one or more of the above biomarkers is increased after administration of a CXCR4 inhibitor.

In some embodiments, a multi-gene signature score, such as an IFN-γ or CTL signature score, can be used as one “biomarker” in the same grouping as other individual gene biomarkers, to calculate a more predictive gene signature score. In some embodiments, the measuring step comprises isolating RNA from the tissue sample and incubating the tissue sample with a set of probes that are designed to specifically hybridize to gene target regions of the RNA.

### Use of CXCR4 Inhibitors and Immunotherapeutic Agents in Treating Cancer

As described in detail below, it has surprisingly been found that treatment of a cancer, such as metastatic melanoma, in a patient with a CXCR4 inhibitor such as X4P-001, optionally in combination with an immunotherapeutic agent such as pembrolizumab, produces a clinical response cytokine signature that correlates with an anti-cancer response in the patient.

Cancer immunotherapy and targeted therapies, such as with ipilimumab or a PD-1 antagonist or antibody, can produce long-lasting responses against metastatic cancer having a wide range of histologies. However, an improved understanding of how some cancers avoid the immune response is required in order to broaden their applicability. It is difficult to study such mechanisms because the interactions between the immune system and cancer cells are continuous and dynamic, meaning that they evolve over time from the initial establishment of the cancer through development of metastasis, which allows the cancer to avoid the immune system. It is now understood that the use of immunotherapy alone may be hindered or rendered ineffective by primary, adaptive, or acquired resistance mechanisms (“immune escape”) (See, e.g., Sharma, P. et al., Cell 168(4): 707-23 (2017)).

Recent studies demonstrate that CXCR4/CXCL12 is a primary receptor-ligand pair that cancer cells and surrounding stromal cells use to block normal immune function and promote angiogenesis through the trafficking of T-effector and T-regulatory cells, as well as myeloid derived suppressor cells (MDSCs), in the tumor microenvironment. Cancer cell CXCR4 overexpression contributes to tumor growth, invasion, angiogenesis, metastasis, relapse, and therapeutic resistance. Accordingly, CXCR4 antagonism represents a means to disrupt tumor-stromal interactions, sensitize cancer cells to cytotoxic drugs, and/or reduce tumor growth and metastatic burden.

CXCR4 (C—X—C chemokine receptor type 4) is a chemokine receptor expressed on a wide range of cell types, including normal stem cells, hematopoietic stem cells (HSC), mature lymphocytes, and fibroblasts (Ratajczak, M. Z. et al. Leukemia 20(11): 1915-24 (2006)). CXCL12 (previously referred to as SDF-1a) is the sole ligand for CXCR4. The primary physiologic functions of the CXCL12/CXCR4 axis include the migration of stem cells both during embryonic development (CXCR4−/− knock-out embryos die in utero) and subsequently in response to injury and inflammation. Increasing evidence indicates multiple potential roles for CXCR4/CXCL12 in malignancy. Direct expression of one or both factors has been observed in several tumor types. CXCL12 is expressed by cancer-associated fibroblast (CAFs) and is often present at high levels in the TME. In clinical studies of a wide range of tumor types, including breast, ovarian, renal, lung, and melanoma, expression of CXCR4/CXCL12 has been associated with a poor prognosis and with an increased risk of metastasis to lymph nodes, lung, liver and brain, which are sites of CXCL12 expression (Scala et al. Clin. Can. Res. 11(5): 1835-41 (2005)). CXCR4 is frequently expressed on melanoma cells, particularly the CD133+population that is considered to represent melanoma stem cells (Scala, S. et al.; Toyozawa, S. et al. Acta Histochem Cytochem 45(5): 293-99 (2012)), and in vitro experiments and murine models have demonstrated that CXCL12 is chemotactic for those cells (Kim, M. et al. Can. Res. 70(24):10411-21 (2010)).

Pembrolizumab is a humanized IgG4 kappa monoclonal antibody that blocks the interaction between PD-1 and its ligands, PD-L1 and PD-L2 [11]. It belongs to the emerging class of immunotherapeutics referred to as checkpoint modulators (CPM). These agents have been developed based on observations that in multiple types of malignancies, the tumor suppresses the host anti-tumor immune response by exploiting counter-regulatory mechanism that normally act as “checkpoints” to prevent the overactivation of the immune system in infection and other situations. In the case of melanoma, PD-L1 is expressed by cells in the TME, engages PD-1, a membrane-associated receptor on CD8⁺ effector T cells, and triggers inhibitory signaling that reduces the killing capacity of cytotoxic T cells.

Pembrolizumab is currently FDA approved for the treatment of unresectable or metastatic melanoma. In a Phase 3 trial, the objective response rate was 33% compared to 12% for ipilimumab (P<0.001) [11]. Analysis of tumor samples before and during treatment in an earlier study demonstrated that a clinical response was associated with an increase in the density of CD8⁺ T cells in the tumor parenchyma (center), while disease progression was associated with persistent low levels of those cells [12]. In an autochthonous murine model of pancreatic adenocarcinoma, persistent tumor growth despite administration of anti-PD-L1 was similarly associated failure of tumor-specific cytotoxic T cells to enter the TME despite their presence in the peripheral circulation [7]. This immunosuppressed phenotype was associated with CXCL12 production by CAF. Moreover, administration of a CXCR4 antagonist (AMD3100) induced rapid T-cell accumulation among the cancer cells and, in combination with anti-PD-L1, synergistically decreased tumor growth.

Nivolumab (Opdivo®, BMS-93568/MDX1106; Bristol-Myers Squibb), is a fully human IgG4 monoclonal antibody that acts as an immunomodulator by binding to the programmed cell death 1 (PD-1) receptor and selectively blocking interaction with its ligands PD-L1 and PD-L2. The structure and other properties of nivolumab are specified at http://www.drugbank.ca/drugs/DB09035, accessed on Mar. 14, 2016, the disclosure of which is hereby incorporated herein. Nivolumab is approved for use in treatment of patients with advanced renal cell carcinoma who have received prior anti-angiogenic therapy; as a single agent in certain types of unresectable or metastatic melanoma; in treating unresectable or metastatic melanoma or in combination with ipilimumab in treating unresectable or metastatic melanoma; and for treatment of metastatic non-small cell lung cancer and progression on or after platinum-based chemotherapy. Additionally, nivolumab has been tested or mentioned as a possible treatment in other oncologic indications, including solid tumors; skin melanoma; glioblastoma; glioma; gliosarcoma; astrocytoma; brain cancer; leukemia; acute myeloid leukemia; chronic myeloid leukemia; chronic lymphocytic leukemia; advanced liver cancer or hepatocellular carcinoma; uveal melanoma; prostate cancer; pancreatic neoplasm and pancreatic cancer; bladder cancer; colorectal cancer; myelodysplastic syndrome; Hodgkin Lymphoma; Non-Hodgkin Lymphoma; multiple myeloma; cervical cancer; endometrial cancer; uterine cancer; ovarian cancer and ovarian carcinoma; peritoneal carcinoma; head and neck squamous cell cancer; gastric cancer; esophageal cancer; Kaposi sarcoma; breast neoplasm, breast adenocarcinoma and breast cancer; bone sarcoma; soft tissue sarcoma; meningiomas; and mesothelioma.

In a phase 3 trial of over 800 patients with advanced clear-cell renal-cell carcinoma, for which they had received previous treatment with one or two regimens of antiangiogenic therapy were randomly assigned to receive 3 mg/kg body weight of nivolumab, intravenously every two weeks, or a 10 mg everolimus tablet orally daily. Patients treated with nivolumab exhibited longer median overall survival, decreased hazard ratio for death, and higher objective response rate than those patients treated with nivolumab (25%) compared to everolimus (5%) (P<0.001), with lower incidence of Grade 3 or 4 treatment-related adverse events (Motzer et al. (2015), New England Journal of Medicine, 373:1803-1813).

In its current prescribed labeling for unresectable or metastatic renal cell carcinoma, the recommended course of administration for nivolumab is 3 mg/kg as an intravenous infusion over 60 minutes every two weeks, until disease progression or unacceptable toxicity. In the discretion of the clinician, depending upon individual tolerance, the prescribed dose of nivolumab may be increased, for example, increased in dosage and/or frequency. In the discretion of the clinician, together with the warnings provided with prescribing information, administration of nivolumab may be discontinued, or the dose reduced in the case of significant adverse effects.

Multiple observations implicate the CXCL12/CXCR4 axis in contributing to the lack (or loss) of tumor responsiveness to angiogenesis inhibitors (also referred to as “angiogenic escape”). In animal cancer models, interference with CXCR4 function has been demonstrated to disrupt the tumor microenvironment (TME) and unmask the tumor to immune attack by multiple mechanisms, including eliminating tumor re-vascularization [19, 20] and increasing the ratio of CD8⁺ T cells to T_{reg} cells [19, 21,22]. These effects result in significantly decreased tumor burden and increased overall survival in xenograft, syngeneic, as well as transgenic, cancer models [19, 20, 21].

X4P-001, formerly designated AMD11070, is a potent, orally bioavailable CXCR4 antagonist [23], that has demonstrated activity in solid and liquid tumor models [24, and unpublished data] and has previously (under the designations AMD070 and AMD11070) been in Phase 1 and 2a trials involving a total of 71 healthy volunteers [23,25,26] and HIV-infected subjects [27,28]. These studies demonstrated that oral administration of up to 400 mg BID for 3.5 days (healthy volunteers) and 200 mg BID for 8-10 days (healthy volunteers and HIV patients) was well-tolerated with no pattern of adverse events or clinically significant laboratory changes. These studies also demonstrated pharmacodynamic activity, with dose- and concentration-related changes in circulating white blood cells (WBCs); and a high volume of distribution (VL), suggesting high tissue penetrance.

Plerixafor (formerly designated AMD3100, now marketed as Mozobil®) is the only CXCR4 antagonist that is currently FDA approved. Plerixafor is administered by subcutaneous injection and is approved for use in combination with granulocyte-colony stimulating factor (G-CSF) to mobilize hematopoietic stem cells (HSCs) to the peripheral blood for collection and subsequent autologous transplantation in patients with non-Hodgkin's lymphoma (NHL) and multiple myeloma (MM).

Both X4P-001 and plerixafor have been studied in murine models of melanoma, renal cell carcinoma, and ovarian cancer and have demonstrated significant anti-tumor activity, including decreased metastasis and increased overall survival [6]. The treatment effect has been associated with decreased presence of myeloid-derived suppressor cells (MDSCs) in the TME and increased presence of tumor-specific CD8⁺ effector cells [7, 8].

In some embodiments, the CXCR4 inhibitor is selected from plerixafor; USL-311 (U.S. Pat. No. 9,353,086), Ulocuplumab (BMS-936564; Kashyap, M. K. et al. Oncotarget 7: 2809-22 (2016)), BL-8040 (BKT-140; Mukhta, E. et al. Mol. Cancer. Ther. 13(2): 275-84 (2014)), T-140 (Jacobson, O. et al. Nuclear Med. 51(11): 1796-1804 (2010), Tamamura, H. et al. FEBS 569: 99-104 (2004)), LY2510924 (Galsky, M. D. et al. Clin. Cancer Res. 20(13): 3581-88 (2014)), TG-0054 (burixafor; NCT00822341), POL6326 (balixafortide; NCT01905475), PRX177561 (Gravina, G. L. et al. Tumor Biol. 39(6):1-17 (2017)), PF-06747143 (Zhang, Y. et al. Sci. Rep. 7: 7305 (2017)), Compound 3 and others (Li, Z. et al. Eur. J. Med. Chem. 149: 30-44 (2017)), GMI-1359 (WO 2016/089872), Compounds Iq, IIj, and others (Bai, R. et al. Eur. J. Med. Chem. 136: 360-71 (2017)), Compound 49b and others (Zhao, H. et al. Bio. Med. Chem Lett. 25(21): 4950-55 (2015)), and F-50067 (515H7; 22nd EORTC-NCI-AACR Symp Molecular Targ Cancer Ther (Berlin), 2010, Abs 225 & 241).

Without wishing to be bound by any particular theory, it is believed that administration of X4P-001 will increase the density of CD8⁺ T cells among the melanoma tumor cells and that this effect will be sustained when X4P-001 is given in combination with an additional cancer therapy such as an immune checkpoint modulator, e.g., pembrolizumab. Because X4P-001 is well-tolerated in the body, and may increase the ability of the body to mount a robust anti-tumor immune response, administering X4P-001 in combination with an additional cancer therapy such as a checkpoint modulator in multiple tumor types may substantially increase the objective response rate, the frequency of durable long-term responses, and overall survival.

It is further believed that such a result would be achieved with comparatively little toxicity since CXCR4-targeted drugs would not be expected to induce cell cycle arrest in bone marrow and other normal proliferating cell populations. Accordingly, the present invention provides significant advantages in treatment outcomes utilizing the low toxicity and effects of the CXCR4 inhibitor X4P-001 on MDSC trafficking, differentiation, and tumor cell gene expression in certain cancers.

CXCR4 antagonism, e.g., by X4P-001, may be used to treat patients with advanced melanoma and other cancers by multiple mechanisms. See WO2017/127811, which is hereby incorporated by reference. In certain embodiments, administration of X4P-001 increases the density of CD8⁺ T cells, thereby resulting in increased anti-tumor immune attack, for example via T cell infiltration of a tumor such as a melanoma tumor. In certain embodiments, administration of X4P-001 additionally decreases neoangiogenesis and tumor vascular supply; and interferes with the autocrine effect of increased expression by tumors of both CXCR4 and its only ligand, CXCL12, thereby potentially reducing cancer cell metastasis.

In some embodiments, patients with advanced forms of cancer, including melanoma, such as metastatic melanoma, or lung cancer, such as metastatic non-small cell lung cancer, are treated with X4P-001, either as a single agent (monotherapy), or in combination with an immune checkpoint inhibitor, such as pembrolizumab. Pembrolizumab is an antibody to PD-1, which binds to the programmed cell death 1 receptor (PD-1), preventing the receptor from binding to the inhibitory ligand PD-L1, and overrides the ability of tumors to suppress the host anti-tumor immune response, dubbed an immune checkpoint inhibitor.

Without wishing to be bound by any particular theory, it is believed that by combining the two medicaments, the patients' treatment outcome can be further improved by increasing the body's ability to mount a robust anti-tumor immune response.

In one aspect, the present invention provides a method of selecting or predicting which melanoma patients from a general population of such patients will be likely (e.g., more likely than average) to benefit from treatment with X4P-001, or a pharmaceutically acceptable salt thereof or pharmaceutical composition thereof, optionally in combination with a checkpoint inhibitor such as pembrolizumab. In some embodiments, the method includes co-administering simultaneously or sequentially an effective amount of one or more additional therapeutic agents, such as those described herein. In some embodiments, the method includes co-administering one additional therapeutic agent. In some embodiments, the method includes co-administering two additional therapeutic agents. In some embodiments, the combination of X4P-001 and the additional therapeutic agent or agents acts synergistically to prevent or reduce immune escape and/or angiogenic escape of the cancer. In some embodiments, the patient has previously been administered another anticancer agent, such as an adjuvant therapy or immunotherapy. In some embodiments, the cancer is refractory. In some embodiments, the additional therapeutic agent is pembrolizumab.

The benefit of neoadjuvant chemo- and immunotherapy has been demonstrated in several operable cancers. Compared to adjuvant therapy, neoadjuvant therapy in patients with locally and regionally advanced cancer has several potential benefits, such as (1) reducing the size of the primary and metastatic tumor increases the probability of achieving negative margin resection; (2) tumor exposure to potentially effective systemic therapy is increased while blood and lymphatic vessels remain intact; and (3) collection of pre- and intra-operative samples of tumor tissue following neoadjuvant therapy offers real-time, in vivo assessment of the effects of the therapy on the tumor cells, the tumor microenvironment (TME), and the immune system.

In some embodiments, X4P-001, or a pharmaceutically acceptable salt thereof, is administered to a patient in a fasted state.

In some embodiments, the present invention provides a method for treating patients with cancer that presents as a solid tumor, such as melanoma. In some embodiments, the patient has resectable melanoma, meaning that the patient's melanoma is deemed susceptible to being removed by surgery. In other embodiments, the patient has unresectable melanoma, meaning that it has been deemed not susceptible to being removed by surgery.

In some embodiments, the present invention provides a method for treating advanced cancer, such as melanoma or metastatic melanoma, in a patient in need thereof, comprising administering X4P-001, or a pharmaceutically acceptable salt and/or composition thereof. In certain embodiments, the patient was previously administered an immune checkpoint inhibitor. In some embodiments, the patient was previously administered an immune checkpoint inhibitor selected from the group consisting of pembrolizumab (Keytruda®, Merck), ipilumumab (Yervoy®, Bristol-Myers Squibb); nivolumab (Opdivo®, Bristol-Myers Squibb) and atezolizumab (Tecentriq®, Genentech). In some embodiments, the cancer became refractory after treatment with the immune checkpoint inhibitor. In some embodiments, the cancer is refractory or resistant to the immune checkpoint inhibitor even though the patient was not previously administered the checkpoint inhibitor. In some embodiments, the cancer is refractory or resistant to PD-1 inhibitors.

In certain embodiments, X4P-001 is co-administered with an immune checkpoint inhibitor, such as those described herein. In some embodiments, the immune checkpoint inhibitor is selected from a PD-1 antagonist, a PD-L1 antagonist, and a CTLA-4 antagonist. In some embodiments, X4P-001 is administered in combination with an immunotherapeutic drug selected from ipilimumab (Yervoy®, Bristol-Myers Squibb); atezolizumab (Tecentriq®, Genentech); nivolumab (Opdivo®, Bristol-Myers Squibb); pidilizumab; avelumab (Bavencio®, Pfizer/Merck KgA); durvalumab (Imfinzi®, AstraZeneca); PDR001; REGN2810; or pembrolizumab (Keytruda®, Merck; previously known as MK-3475). In some embodiments, X4P-001 is administered in combination with pembrolizumab.

Other immune checkpoint inhibitors in development may also be suitable for use in combination with X4P-001. These include atezolizumab (Tecentriq®, Genentech/Roche), also known as MPDL3280A, a fully humanized engineered antibody of IgG1 isotype against PD-L1, in clinical trials for non-small cell lung cancer, and advanced bladder cancer, such as advanced urothelial carcinoma; and as adjuvant therapy to prevent cancer from returning after surgery; durvalumab (Astra-Zeneca), also known as MEDI4736, in clinical trials for metastatic breast cancer, multiple myeloma, esophageal cancer, myelodysplastic syndrome, small cell lung cancer, head and neck cancer, renal cancer, glioblastoma, lymphoma and solid malignancies; pidilizumab (CureTech), also known as CT-011, an antibody that binds to PD-1, in clinical trials for diffuse large B-cell lymphoma and multiple myeloma; avelumab (Pfizer/Merck KGaA), also known as MSB0010718C, a fully human IgG1 anti-PD-L1 antibody, in clinical trials for non-small cell lung cancer, Merkel cell carcinoma, mesothelioma, solid tumors, renal cancer, ovarian cancer, bladder cancer, head and neck cancer and gastric cancer; and PDR001 (Novartis), an inhibitory antibody that binds to PD-1, in clinical trials for non-small cell lung cancer, melanoma, triple negative breast cancer and advanced or metastatic solid tumors.

Other immune checkpoint inhibitors suitable for use in the present invention include REGN2810 (Regeneron), an anti-PD-1 antibody tested in patients with basal cell carcinoma (NCT03132636); NSCLC (NCT03088540); cutaneous squamous cell carcinoma (NCT02760498); lymphoma (NCT02651662); and melanoma (NCT03002376); pidilizumab (CureTech), also known as CT-011, an antibody that binds to PD-1, in clinical trials for diffuse large B-cell lymphoma and multiple myeloma; avelumab (Bavencio®, Pfizer/Merck KGaA), also known as MSB0010718C), a fully human IgG1 anti-PD-L1 antibody, in clinical trials for non-small cell lung cancer, Merkel cell carcinoma, mesothelioma, solid tumors, renal cancer, ovarian cancer, bladder cancer, head and neck cancer, and gastric cancer; and PDR001 (Novartis), an inhibitory antibody that binds to PD-1, in clinical trials for non-small cell lung cancer, melanoma, triple negative breast cancer and advanced or metastatic solid tumors. Tremelimumab (CP-675,206; Astrazeneca) is a fully human monoclonal antibody against CTLA-4 that has been in studied in clinical trials for a number of indications, including: mesothelioma, colorectal cancer, kidney cancer, breast cancer, lung cancer and non-small cell lung cancer, pancreatic ductal adenocarcinoma, pancreatic cancer, germ cell cancer, squamous cell cancer of the head and neck, hepatocellular carcinoma, prostate cancer, endometrial cancer, metastatic cancer in the liver, liver cancer, large B-cell lymphoma, ovarian cancer, cervical cancer, metastatic anaplastic thyroid cancer, urothelial cancer, fallopian tube cancer, multiple myeloma, bladder cancer, soft tissue sarcoma, and melanoma. AGEN-1884 (Agenus) is an anti-CTLA4 antibody that is being studied in Phase 1 clinical trials for advanced solid tumors (NCT02694822).

Pembrolizumab (Keytruda®, Merck) is a humanized antibody that targets the programmed cell death (PD-1) receptor. The structure and other properties of pembrolizumab are specified at http://www.drugbank.ca/drugs/DB09037, accessed on Jan. 18, 2016, the disclosure of which is hereby incorporated herein. Pembrolizumab is approved for use in treating unresectable melanoma and metastatic melanoma, and metastatic non-small cell lung cancer in patients whose tumors express PD-1, and have failed treatment with other chemotherapeutic agents. Additionally, pembrolizumab has been tested or mentioned as a possible treatment in other oncologic indications, including solid tumors, thoracic tumors, thymic epithelial tumors, thymic carcinoma, leukemia, ovarian cancer, esophageal cancer, small cell lung cancer, head and neck cancer, salivary gland cancer, colon cancer, rectal cancer, colorectal cancer, urothelial cancer, endometrial cancer, bladder cancer, cervical cancer, hormone-resistant prostate cancer, testicular cancer, triple negative breast cancer, renal cell and kidney cancer, pancreatic adenocarcinoma and pancreatic cancer, gastric adenocarcinoma, gastrointestinal and stomach cancer; brain tumor, malignant glioma, glioblastoma, neuroblastoma, lymphoma, sarcoma, mesothelioma, respiratory papilloma, myelodysplastic syndrome and multiple myeloma.

In a Phase 3 trial in unresectable or metastatic melanoma, the objective response rate was 33% compared to 12% for ipilimumab (P<0.001) [11]. Analysis of tumor samples before and during treatment in an earlier study demonstrated that a clinical response was associated with an increase in the density of CD8⁺ T cells in the tumor parenchyma (center), while disease progression was associated with persistent low levels of those cells [12]. In an autochthonous murine model of pancreatic adenocarcinoma, persistent tumor growth despite administration of anti-PD-L1 was similarly associated failure of tumor-specific cytotoxic T cells to enter the TME despite their presence in the peripheral circulation [7]. This immunosuppressed phenotype was associated with CXCL12 production by CAF. By increasing the density of CD8⁺ T cells among the melanoma tumor cells administration of X4P-001 in combination with pembrolizumab or other checkpoint modulators in multiple tumor types may substantially increase the objective response rate, the frequency of durable long-term responses, and overall survival.

In its current prescribed labeling for unresectable or metastatic melanoma, the recommended course of administration for pembrolizumab is 2 mg/kg as an intravenous infusion over 30 minutes every three weeks. In the discretion of the clinician, depending upon individual tolerance, the prescribed dose of pembrolizumab may be increased to 10 mg/kg every 21 days or 10 mg/kg every 14 days. In the discretion of the clinician, together with the warnings provided with prescribing information, administration of pembrolizumab may be discontinued, or the dose reduced in the case of significant adverse effects.

In some embodiments, the present invention provides a method for treating metastatic melanoma in a patient comprising administering to the patient X4P-001 or a pharmaceutically acceptable salt thereof in combination with an immune checkpoint inhibitor. In some embodiments, the melanoma is resectable and metastatic. In other embodiments, the melanoma is unresectable and metastatic. In some embodiments, the immune checkpoint inhibitor is pembrolizumab.

In some embodiments, the present invention provides a method for treating resectable metastatic melanoma in a patient comprising administering to the patient X4P-001 or a pharmaceutically acceptable salt thereof in combination with an immune checkpoint inhibitor. After completion of treatment in accordance with the present invention, resection surgery may be performed. In other embodiments, the present invention provides a method for treating unresectable metastatic melanoma in a patient comprising administering to the patient X4P-001 or a pharmaceutically acceptable salt thereof in combination with an immune checkpoint inhibitor. In some embodiments, the immune checkpoint inhibitor is pembrolizumab. After completion of treatment in accordance with the present invention, the patient may continue to receive standard of care (SOC) therapy with pembrolizumab or another therapy per the treating clinician's discretion, and such treatment may include further treatment with X4P-001 or a pharmaceutically acceptable salt thereof.

In some embodiments, the present invention provides a method for treating a refractory cancer in a patient in need thereof, wherein said method comprises administering to said patient X4P-001 or a pharmaceutically acceptable salt thereof in combination with an immune checkpoint inhibitor. In some embodiments, the refractory cancer is metastatic melanoma that expresses PD-L1. In some embodiments, the metastatic melanoma expresses PD-L1 and exhibits disease progression after the patient has undergone chemotherapy or treatment with an immune checkpoint inhibitor but not X4P-001. In some embodiments, the refractory cancer is metastatic non-small cell lung cancer (NSCLC) that expresses PD-L1, and which exhibits disease progression after platinum-containing chemotherapy. In some embodiments, the refractory cancer is metastatic melanoma and the immune checkpoint inhibitor is pembrolizumab.

In some embodiments, a provided method comprises administering X4P-001, or a pharmaceutically acceptable salt thereof, to a patient in a fasted state and administering the immune checkpoint inhibitor to a patient in either a fasted or fed state.

In certain embodiments, the present invention provides a method for treating cancer in a patient in need thereof, wherein said method comprises administering to said patient X4P-001 or a pharmaceutically acceptable salt thereof in combination with an immune checkpoint inhibitor, further comprising the step of obtaining a biological sample from the patient and measuring the amount of a disease-related biomarker. In some embodiments, the biological sample is a blood sample or skin punch biopsy.

In certain embodiments, the disease-related biomarker is a cytokine panel, a cytokine signature, a ratio of one or more cytokine ratios, or a cytokine score.

In certain embodiments, the disease-related biomarker is circulating CD8⁺ T cells and/or plasma levels of PD-1 and/or PD-L1. In some embodiments, the biomarker one or more of is CD8⁺ T cells or CD8⁺ T cells/T_{reg} ratio, granzyme B, an IFN-γ signature score, a CTL signature score, an antigen presentation/processing signature score, a tumor inflammation signature score, or PD-L1 expression.

In some embodiments, the biomarker is a cytokine panel. In some embodiments, the cytokine panel comprises one or more biomarkers selected from ANG-1, ENA-78, Latency-associated peptide of transforming growth factor beta 1, MCP-1, and PDGF-BB. In some embodiments, the expression level of one or more of the above biomarkers is decreased after administration of a CXCR4 inhibitor.

In some embodiments, the cytokine panel comprises one or more biomarkers selected from 6CKine, Decorin, IL-2, MIP-3 beta, MIG (CXCL9), and MPIF-1P. In some embodiments, the expression level of one or more of the above biomarkers is increased after administration of a CXCR4 inhibitor.

In certain embodiments, the present invention provides a method for treating advanced cancer, such as melanoma or non-small cell lung cancer, in a patient in need thereof, wherein the method comprises administering to said patient X4P-001 or a pharmaceutically acceptable salt thereof in combination with pembrolizumab, further comprising the step of obtaining a biological sample from the patient and measuring the amount of a disease-related biomarker. In some embodiments, the biological sample is a blood sample or skin punch biopsy. In certain embodiments, the disease-related biomarker is a cytokine panel, a cytokine signature, a ratio of one or more cytokine ratios, or a cytokine score. In certain embodiments, the disease-related biomarker is circulating CD8⁺ cells and/or plasma levels of PD-1 and/or PD-L1. In some embodiments, the disease-related biomarker is one or more of CD8⁺ T cells or CD8⁺ T cells/T_{reg} ratio, granzyme B, an IFN-γ signature score, a CTL signature score, an antigen presentation/processing signature score, a tumor inflammation signature score, or PD-L1 expression.

In some embodiments, the biomarker is a cytokine panel. In some embodiments, the cytokine panel comprises one or more biomarkers selected from ANG-1, ENA-78, Latency-associated peptide of transforming growth factor beta 1, MCP-1, and PDGF-BB. In some embodiments, the expression level of one or more of the above biomarkers is decreased after administration of a CXCR4 inhibitor.

In some embodiments, the cytokine panel comprises one or more biomarkers selected from 6CKine, Decorin, IL-2, MIP-3 beta, MIG (CXCL9), and MPIF-1P. In some embodiments, the expression level of one or more of the above biomarkers is increased after administration of a CXCR4 inhibitor.

In other embodiments of the invention, X4P-001 or a pharmaceutically acceptable salt thereof is administered in combination with an immune checkpoint inhibitor. The immune checkpoint inhibitor may be an antibody to PD-1, PD-L1, or CTLA-4. In certain embodiments, the immune checkpoint antagonist is selected from pembrolizumab, nivolumab, and ipilimumab.

In some embodiments, the present invention provides a method of treating cancer in a patient in need thereof, wherein said method comprises administering to said patient X4P-001 or a pharmaceutically acceptable salt thereof in combination with an immune checkpoint inhibitor, wherein the X4P-001 or a pharmaceutically acceptable salt thereof and the immune checkpoint inhibitor act synergistically. One of ordinary skill in the art will appreciate that active agents (such as X4P-001 and an immune checkpoint inhibitor) act synergistically when the combination of active agents results in an effect that is greater than additive. In some embodiments, the immune checkpoint inhibitor is pembrolizumab.

In some embodiments, the present invention provides a method for sensitizing a cancer in a patient in need thereof, wherein the method comprises administering to said patient a CXCR4 inhibitor, such as X4P-001 or a pharmaceutically acceptable salt thereof, in combination with an immune checkpoint inhibitor. In some embodiments, the method comprises administering X4P-001 to the patient prior to treatment with the immune checkpoint inhibitor. In some embodiments, the cancer is a solid tumor. In some embodiments, the method comprises first obtaining from the patient a tumor sample, such as a biopsy of the patient's cancer or solid tumor, a baseline measurement of a biomarker for sensitivity to treatment with an immune checkpoint inhibitor, and comparing the baseline measurement to a pre-established threshold for treatment with an immune checkpoint inhibitor. In a case where the baseline measurement does not meet the pre-established threshold of the biomarker for sensitivity to treatment with an immune checkpoint inhibitor, the patient is treated with a CXCR4 inhibitor such as X4P-001 or a pharmaceutically acceptable salt thereof, with the desired effect of altering (e.g., increasing or decreasing, as the case may be) the baseline measurement to achieve an altered measurement that meets the pre-established threshold. After the patient has been treated with X4P-001 or a pharmaceutically acceptable salt thereof, and found to meet the pre-established threshold, the patient is subsequently treated with an immune checkpoint inhibitor, such as a PD-1 inhibitor or a PD-L1 inhibitor.

It is also within the present invention for the treating clinician, in his or her discretion, to treat the patient with an immune checkpoint inhibitor, even if the patient's altered measurement does not meet the pre-established threshold, if it is considered that the patient may still benefit from treatment with the immune checkpoint inhibitor. Alternatively, the treating clinician may continue to treat the patient with X4P-001 or a pharmaceutically acceptable salt thereof, and continue to monitor the patient's biomarker levels to achieve the pre-established threshold. It is also within the present invention for the treating clinician, in his or her discretion, to alter the treatment plan for the patient, or to discontinue treatment altogether.

Immune checkpoint inhibitors of use in the present invention include, for example, pembrolizumab, nivolumab, atezolizumab, avelumab, durvalumab, ipilumumab, and pidilizumab.

In certain embodiments, the biomarker is PD-L1. In other embodiments, the biomarker comprises a gene signature for a relevant pathway or gene. In certain embodiments, the biomarker comprises a gene signature for interferon gamma (IFN-γ), which may be a gene signature based upon the expression levels some or all of the genes selected from IFN-γ, CXCL9, CXCL10, HLA-DRA, IDOL or STAT1. In some embodiments, the gene signature comprises all six genes IFN-γ, CXCL9, CXCL10, HLA-DRA, IDO1, and STAT1. In certain embodiments, the pre-established threshold has been incorporated into the prescribing information that is included in the package insert, on the packaging, or on a website associated with the CXCR4 inhibitor or said immune checkpoint inhibitor.

A variety of cancers may be treated as provided by the present invention. In some embodiments, the cancer is selected from hepatocellular carcinoma, ovarian cancer, ovarian epithelial cancer, fallopian tube cancer; papillary serous cystadenocarcinoma or uterine papillary serous carcinoma (UPSC); prostate cancer; testicular cancer; gallbladder cancer; hepatocholangiocarcinoma; soft tissue and bone synovial sarcoma; rhabdomyosarcoma; osteosarcoma; chondrosarcoma; Ewing sarcoma; anaplastic thyroid cancer; adrenocortical adenoma; pancreatic cancer; pancreatic ductal carcinoma or pancreatic adenocarcinoma; gastrointestinal/stomach (GIST) cancer; lymphoma; squamous cell carcinoma of the head and neck (SCCHN); salivary gland cancer; glioma, or brain cancer; neurofibromatosis-1 associated malignant peripheral nerve sheath tumors (MPNST); Waldenstrom's macroglobulinemia; or medulloblastoma.

In some embodiments, the cancer is selected from hepatocellular carcinoma (HCC), hepatoblastoma, colon cancer, rectal cancer, ovarian cancer, ovarian epithelial cancer, fallopian tube cancer, papillary serous cystadenocarcinoma, uterine papillary serous carcinoma (UPSC), hepatocholangiocarcinoma, soft tissue and bone synovial sarcoma, rhabdomyosarcoma, osteosarcoma, anaplastic thyroid cancer, adrenocortical adenoma, pancreatic cancer, pancreatic ductal carcinoma, pancreatic adenocarcinoma, glioma, neurofibromatosis-1 associated malignant peripheral nerve sheath tumors (MPNST), Waldenstrom's macroglobulinemia, or medulloblastoma.

In some embodiments, the present invention provides a method for treating a cancer that presents as a solid tumor, such as a sarcoma, carcinoma, or lymphoma, comprising the step of administering X4P-001, or a pharmaceutically acceptable salt thereof, to a patient in need thereof. Solid tumors generally comprise an abnormal mass of tissue that typically does not include cysts or liquid areas. In some embodiments, the cancer is selected from renal cell carcinoma, or kidney cancer; hepatocellular carcinoma (HCC) or hepatoblastoma, or liver cancer; melanoma; breast cancer; colorectal carcinoma, or colorectal cancer; colon cancer; rectal cancer; anal cancer; lung cancer, such as non-small cell lung cancer (NSCLC) or small cell lung cancer (SCLC); ovarian cancer, ovarian epithelial cancer, ovarian carcinoma, or fallopian tube cancer; papillary serous cystadenocarcinoma or uterine papillary serous carcinoma (UPSC); prostate cancer; testicular cancer; gallbladder cancer; hepatocholangiocarcinoma; soft tissue and bone synovial sarcoma; rhabdomyosarcoma; osteosarcoma; chondrosarcoma; Ewing sarcoma; anaplastic thyroid cancer; adrenocortical carcinoma; pancreatic cancer; pancreatic ductal carcinoma or pancreatic adenocarcinoma; gastrointestinal/stomach (GIST) cancer; lymphoma; squamous cell carcinoma of the head and neck (SCCHN); salivary gland cancer; glioma, or brain cancer; neurofibromatosis-1 associated malignant peripheral nerve sheath tumors (MPNST); Waldenstrom's macroglobulinemia; or medulloblastoma.

In some embodiments, the cancer is selected from renal cell carcinoma, hepatocellular carcinoma (HCC), hepatoblastoma, colorectal carcinoma, colorectal cancer, colon cancer, rectal cancer, anal cancer, ovarian cancer, ovarian epithelial cancer, ovarian carcinoma, fallopian tube cancer, papillary serous cystadenocarcinoma, uterine papillary serous carcinoma (UPSC), hepatocholangiocarcinoma, soft tissue and bone synovial sarcoma, rhabdomyosarcoma, osteosarcoma, chondrosarcoma, anaplastic thyroid cancer, adrenocortical carcinoma, pancreatic cancer, pancreatic ductal carcinoma, pancreatic adenocarcinoma, glioma, brain cancer, neurofibromatosis-1 associated malignant peripheral nerve sheath tumors (MPNST), Waldenstrom's macroglobulinemia, or medulloblastoma.

In some embodiments, the cancer is renal cell carcinoma (RCC) or clear cell renal carcinoma (ccRCC).

In some embodiments, the cancer is selected from hepatocellular carcinoma (HCC), hepatoblastoma, colon cancer, rectal cancer, ovarian cancer, ovarian epithelial cancer, ovarian carcinoma, fallopian tube cancer, papillary serous cystadenocarcinoma, uterine papillary serous carcinoma (UPSC), hepatocholangiocarcinoma, soft tissue and bone synovial sarcoma, rhabdomyosarcoma, osteosarcoma, anaplastic thyroid cancer, adrenocortical carcinoma, pancreatic cancer, pancreatic ductal carcinoma, pancreatic adenocarcinoma, glioma, neurofibromatosis-1 associated malignant peripheral nerve sheath tumors (MPNST), Waldenstrom's macroglobulinemia, or medulloblastoma.

In some embodiments, the cancer is hepatocellular carcinoma (HCC). In some embodiments, the cancer is hepatoblastoma. In some embodiments, the cancer is colon cancer. In some embodiments, the cancer is rectal cancer. In some embodiments, the cancer is ovarian cancer, or ovarian carcinoma. In some embodiments, the cancer is ovarian epithelial cancer. In some embodiments, the cancer is fallopian tube cancer. In some embodiments, the cancer is papillary serous cystadenocarcinoma. In some embodiments, the cancer is uterine papillary serous carcinoma (UPSC). In some embodiments, the cancer is hepatocholangiocarcinoma. In some embodiments, the cancer is soft tissue and bone synovial sarcoma. In some embodiments, the cancer is rhabdomyosarcoma. In some embodiments, the cancer is osteosarcoma. In some embodiments, the cancer is anaplastic thyroid cancer. In some embodiments, the cancer is adrenocortical carcinoma. In some embodiments, the cancer is pancreatic cancer, or pancreatic ductal carcinoma. In some embodiments, the cancer is pancreatic adenocarcinoma. In some embodiments, the cancer is glioma. In some embodiments, the cancer is malignant peripheral nerve sheath tumors (MPNST). In some embodiments, the cancer is neurofibromatosis-1 associated MPNST. In some embodiments, the cancer is Waldenstrom's macroglobulinemia. In some embodiments, the cancer is medulloblastoma.

In some embodiments, the present invention provides a method for treating a cancer selected from leukemia or a cancer of the blood, comprising administering to a patient in need thereof an effective amount of X4P-001 or a pharmaceutically acceptable salt thereof or pharmaceutical composition thereof, optionally in combination with an additional therapeutic agent such as those described herein. In some embodiments, the cancer is selected from acute myeloid leukemia (AML), chronic myeloid leukemia (CML), acute lymphocytic leukemia (ALL), chronic lymphocytic leukemia (CLL), or a virally induced leukemia.

In some embodiments, the patient has a resectable solid tumor, meaning that the patient's tumor is deemed susceptible to being removed by surgery. In other embodiments, the patient has an unresectable solid tumor, meaning that the patient's tumor has been deemed not susceptible to being removed by surgery, in whole or in part.

In some embodiments, the cancer is an advanced cancer, such as an advanced kidney cancer or advanced renal cell carcinoma.

### Disease-Related Biomarkers

Cancer research is improved by the identification of intratumoral expression patterns for sets of genes, changes in levels of immune-related cells in the tumor microenvironment, or other changes in the tumor microenvironment, referred to herein generally as “biomarkers” or more specifically in relation to gene expression patterns as “gene signatures,” “gene expression biomarkers,” or “molecular signatures,” which are characteristic of particular types or subtypes of cancer, and which are associated with clinical outcomes. If such an association is predictive of a clinical response, the biomarker is advantageously used in methods of selecting or stratifying patients as more (or less, as the case may be) likely to benefit from a treatment regimen disclosed herein. It has now been surprisingly found that levels of serum cytokines, and ratios thereof, may be used as biomarkers in a method described herein, such as a method of treating cancer in a patient, diagnosing a cancer in a patient, or predicting patient response to treatment of a cancer such as metastatic melanoma.

It has been surprisingly found that X4P-001 increases levels of serum CXCL9 and CXCL10 in patients with cancers such as solid tumors, e.g., advanced or metastatic melanoma. CXCL9 is a known as a T cell chemoattractant. CXCL10 is known as a T cell chemoattractant and an inhibitor of angiogenesis. Accordingly, in some embodiments, the biomarker is an observed increase in serum CXCL9 and/or CXCL10 in a tumor relative to a control. In some embodiments, the biomarker is a change in the ratio between CXCL9 and CXCL10. In some embodiments, the cancer is a solid tumor such as advanced or metastatic melanoma. In some embodiments, the cancer is melanoma, RCC, or ccRCC.

In some embodiments, the biomarker comprises a change in the serum concentration of CXCL9 and/or CXCL10 in a patient after treatment, such as after 1, 2, 3, 4, 5, 6, 7, 8, 9, or more weeks of treatment. In some embodiments, the serum concentration of CXCL9 is increased after treatment by at least about 1.0-fold. In some embodiments, the serum concentration of CXCL9 is increased after treatment by at least about 1.5-fold. In some embodiments, the serum concentration of CXCL9 is increased after treatment by at least about 2.0-fold. In some embodiments, the serum concentration of CXCL9 is increased after treatment by at least about 2.5-fold. In some embodiments, the serum concentration of CXCL9 is increased after treatment by at least about 3.0-fold. In some embodiments, the serum concentration of CXCL9 is increased after treatment by at least about 3.5-fold. In some embodiments, the serum concentration of CXCL9 is increased after treatment by at least about 4.0-fold. In some embodiments, the serum concentration of CXCL9 is increased after treatment by at least about 4.5-fold. In some embodiments, the serum concentration of CXCL9 is increased after treatment by up to about 5.0-fold. In some embodiments, the serum concentration of CXCL10 is increased after treatment by at least about 1.0-fold. In some embodiments, the serum concentration of CXCL10 is increased after treatment by at least about 1.5-fold. In some embodiments, the serum concentration of CXCL10 is increased after treatment by at least about 2.0-fold. In some embodiments, the serum concentration of CXCL10 is increased after treatment by at least about 2.5-fold. In some embodiments, the serum concentration of CXCL10 is increased after treatment by at least about 3.0-fold. In some embodiments, the serum concentration of CXCL10 is increased after treatment by at least about 3.5-fold. In some embodiments, the serum concentration of CXCL10 is increased after treatment by at least about 4.0-fold. In some embodiments, the serum concentration of CXCL10 is increased after treatment by at least about 4.5-fold. In some embodiments, the serum concentration of CXCL10 is increased after treatment by up to about 5.0-fold. In some embodiments, the treatment is one of those described herein, such as a combination of X4P-001, or a pharmaceutically acceptable salt thereof, and nivolumab or pembrolizumab.

It has been surprisingly found that X4P-001 increases observed numbers of CD8⁺ T cells and/or CD4⁺ T cells in cancers such as solid tumors, e.g., advanced or metastatic melanoma. Accordingly, in some embodiments, the biomarker is an observed increase in CD8⁺ T cells and/or CD4⁺ T cells in a tumor relative to a control. In other embodiments, the biomarker is an increase in the ratio of CD8⁺ T cells to T_{reg} cells. In some embodiments, the increase is observed by immunohistochemistry or expression levels of one or both of CD8A and CD8B. In some embodiments, an increase in CD8⁺ T cells and/or CD4⁺ T cells or CD8⁺ T cells/T_{reg} ratio in a tumor sample from a patient who has undergone treatment with X4P-001 correlates with an increased likelihood that the patient will benefit from continued treatment with X4P-001 alone or in combination with an immunotherapeutic agent, e.g., a checkpoint inhibitor such as a PD-1 antagonist. In some embodiments, the PD-1 antagonist is selected from nivolumab, pembrolizumab, a pembrolizumab biosimilar, or a pembrolizumab variant. In some embodiments, the checkpoint inhibitor is pembrolizumab. In some embodiments, the tumor is a solid tumor such as advanced or metastatic melanoma, or RCC or ccRCC.

It has been surprisingly found that X4P-001 modulates levels of one or more of a panel of serum cytokines referred to herein as a “cytokine panel.” In some embodiments, the cytokine panel comprises a set of biomarkers comprising one or more biomarkers whose expression changes (i.e., increases or decreases) in response to treatment with a CXCR4 inhibitor. In some embodiments, the biomarkers of a cytokine panel comprise one or more of Adiponectin, AXL Receptor Tyrosine Kinase (AXL), Brain-Derived Neurotrophic Factor (BDNF), Carcinoembryonic antigen-related cell adhesion molecule 1 (CEACAM1), Decorin, EN-RAGE, Eotaxin-1, Eotaxin-2, Epidermal Growth Factor Receptor (EGFR), Epidermal Growth Factor (EGF), Epithelial-Derived Neutrophil-Activating Protein 78 (ENA-78), E-Selectin, Factor VII, FASLG Receptor (FAS), Ferritin (FRTN), Growth-Regulated alpha protein (GRO-alpha), Heparin-Binding EGF-Like Growth Factor (HB-EGF), Hepatocyte Growth Factor (HGF), Hepsin, Immunoglobulin E (IgE), Intercellular Adhesion Molecule 1 (ICAM-1), Interferon gamma Induced Protein 10 (IP-10), Interferon gamma Simoa (IFN-gamma Simoa), Interferon-inducible T-cell alpha chemoattractant (ITAC), Interleukin-2 receptor alpha (IL-2 receptor alpha), Latency-Associated Peptide of Transforming Growth Factor beta 1, Macrophage-Derived Chemokine (MDC), Macrophage inflammatory protein 3 beta (MIP-3 beta), Macrophage Inflammatory Protein-1 beta (MIP-1 beta), Macrophage Inflammatory Protein-3 alpha (MIP-3 alpha), Monocyte Chemotactic Protein 1 (MCP-1), Monocyte Chemotactic Protein 2 (MCP-2), Monocyte Chemotactic Protein 4 (MCP-4), Monokine Induced by Gamma Interferon (MIG), Myeloid Progenitor Inhibitory Factor 1 (MPIF-1), Myoglobin, Osteoprotegerin (OPG), Plasminogen Activator Inhibitor 1 (PAI-1), Platelet-Derived Growth Factor BB (PDGF-BB), Platelet endothelial cell adhesion molecule (PECAM-1), Prostate-Specific Antigen Free (PSA-f), Pulmonary and Activation-Regulated Chemokine (PARC), Pulmonary surfactant-associated protein D (SP-D), Stem Cell Factor (SCF), Tissue Inhibitor of Metalloproteinases 1 (TIMP-1), TNF-Related Apoptosis-Inducing Ligand Receptor 3 (TRAIL-R3), Tumor necrosis factor receptor 2 (TNFR2), Tumor Necrosis Factor Receptor I (TNF RI), Urokinase-type plasminogen activator receptor (uPAR), Angiopoietin-1 (ANG-1), B cell-activating factor (BAFF), Cancer Antigen 15-3 (CA-15-3), Carbonic anhydrase 9 (CA-9), Chemokine CC-4 (HCC-4), Interleukin-6 receptor (IL-6r), Interleukin-2 Simoa (IL-2 Simoa), Interleukin-10 (IL-10), Interleukin-16 (IL-16), Interleukin-18 (IL-18), Interleukin-5 Simoa (IL-5 Simoa), Matrix Metalloproteinase-3 (MMP-3), Alpha-2-Macroglobulin (A2Macro), Stromal cell-derived factor-1 (SDF-1), T-Cell-Specific Protein RANTES (RANTES), Tenascin-C(TN-C), Vascular Cell Adhesion Molecule-1 (VCAM-1), Vascular Endothelial Growth Factor Receptor 2 (VEGFR-2), Vascular endothelial growth factor receptor 3 (VEGFR-3), Vascular Endothelial Growth Factor (VEGF), and 6Ckine.

In some embodiments, the cytokine panel comprises one or more biomarkers selected from ANG-1, ENA-78, Latency-associated peptide of transforming growth factor beta 1, MCP-1, and PDGF-BB. In some embodiments, the expression level of one or more of the above biomarkers is decreased after administration of a CXCR4 inhibitor.

In some embodiments, the cytokine panel comprises one or more biomarkers selected from 6CKine, Decorin, IL-2, MIP-3 beta, MIG (CXCL9), and MPIF-1P. In some embodiments, the expression level of one or more of the above biomarkers is increased after administration of a CXCR4 inhibitor.

In some embodiments, the cytokine panel is selected from a change (i.e., an increase or decrease) of one or more of IL-6, IL-7, IL-8, IL-10, IL-12p70, IL-22, IL-23p19, IFN-α2, IFN-γ, TNF-β, monocyte chemoattractant protein-1 (MCP-1), stromal cell-derived factor 1A (SDF-1), interferon gamma-induced protein 10 (IP-10 or CXCL10), monokine induced by interferon gamma (MIG or CXCL9), granulocyte-macrophage colony-stimulating factor (GM-CSF), platelet-derived growth factor (PDGF), hepatocyte growth factor (HGF), and vesicular endothelial growth factor-A (VEGF-A) (Yamazaki, N. et al. Cancer Science 108(5): 1022-31 (2017)). In some embodiments, the cytokine panel is two or more of IL-6, IL-7, IL-8, IL-10, IL-12p70, IL-22, IL-23p19, IFN-α2, IFN-γ, TNF-β, MCP-1, SDF-1, CXCL10, CXCL9, GM-CSF, PDGF, HGF, and VEGF-A. In some embodiments, the cytokine panel is three or more of IL-6, IL-7, IL-8, IL-10, IL-12p70, IL-22, IL-23p19, IFN-α2, IFN-γ, TNF-β, MCP-1, SDF-1, CXCL10, CXCL9, GM-CSF, PDGF, HGF, and VEGF-A. In some embodiments, the cytokine panel is four or more of IL-6, IL-7, IL-8, IL-10, IL-12p70, IL-22, IL-23p19, IFN-α2, IFN-γ, TNF-β, MCP-1, SDF-1, CXCL10, CXCL9, GM-CSF, PDGF, HGF, and VEGF-A. In some embodiments, the cytokine panel is five or more of IL-6, IL-7, IL-8, IL-10, IL-12p70, IL-22, IL-23p19, IFN-α2, IFN-γ, TNF-β, MCP-1, SDF-1, CXCL10, CXCL9, GM-CSF, PDGF, HGF, and VEGF-A. In some embodiments, the cytokine panel is ten or more of IL-6, IL-7, IL-8, IL-10, IL-12p70, IL-22, IL-23p19, IFN-α2, IFN-γ, TNF-β, MCP-1, SDF-1, CXCL10, CXCL9, GM-CSF, PDGF, HGF, and VEGF-A. In some embodiments, the cytokine panel is fifteen or more of IL-6, IL-7, IL-8, IL-10, IL-12p70, IL-22, IL-23p19, IFN-α2, IFN-γ, TNF-β, MCP-1, SDF-1, CXCL10, CXCL9, GM-CSF, PDGF, HGF, and VEGF-A. In some embodiments, the cytokine panel is all of IL-6, IL-7, IL-8, IL-10, IL-12p70, IL-22, IL-23p19, IFN-α2, IFN-γ, TNF-β, MCP-1, SDF-1, CXCL10, CXCL9, GM-CSF, PDGF, HGF, and VEGF-A.

In some embodiments, the cytokine panel is one or more of IFN-γ, CXCL10, and CXCL9. In some embodiments, the cytokine panel is two or more of IFN-γ, CXCL10, and CXCL9. In some embodiments, the cytokine panel is all three of IFN-γ, CXCL10, and CXCL9.

In some embodiments, the cytokine panel comprises one or more biomarkers selected from ANG-1, ENA-78, Latency-associated peptide of transforming growth factor beta 1, MCP-1, and PDGF-BB. In some embodiments, the expression level of one or more of the above biomarkers is decreased after administration of a CXCR4 inhibitor. In some embodiments, the cytokine panel comprises one or more biomarkers selected from 6CKine, Decorin, IL-2, MIP-3 beta, MIG (CXCL9), and MPIF-1P. In some embodiments, the expression level of one or more of the above biomarkers is increased after administration of a CXCR4 inhibitor.

In some embodiments, the biomarkers of a cytokine panel comprise one or more of TNF-related apoptosis-inducing ligand receptor (TRAIL-R3), Interleukin-6 receptor (IL-6r), Myeloid Progenitor Inhibitor Factor (MPIF-1), Tumor Necrosis Factor Receptor 2 (TNFR2), Interleukin-2 Simoa (IL-2 Simoa), Monokine Induced by Gamma Interferon (MIG; CXCL9), EN-RAGE, Tumor Necrosis Factor Receptor 1 (TNF R1), Eotaxin-2, Chemokine CC-4 (HCC-4), Urokinase-type Plasminogen Activator Receptor (uPAR), Interleukin-2 Receptor Alpha (IL-2 receptor alpha), Macrophage Inflammatory Protein-1 beta (MIP-1 beta), Interferon gamma Induced Protein 10 (IP-10; CXCL10), 6Ckine, Macrophage Inflammatory Protein-3 beta (MIP-3 beta), Macrophage-Derived Chemokine (MDC), AXL Receptor Tyrosine Kinase (AXL), Tissue Inhibitor of Metalloproteinases 1 (TIMP-1), Plasminogen Activator Inhibitor 1 (PAI-1), Brain-Derived Neurotrophic Factor (BDNF), Epidermal Growth Factor (EGF), E-Selectin, and Monocyte Chemotactic Protein 2 (MCP-2).

In some embodiments, the biomarkers of a cytokine panel comprise one or more of TNF-related apoptosis-inducing ligand receptor (TRAIL-R3), Interleukin-6 receptor (IL-6r), Myeloid Progenitor Inhibitor Factor (MPIF-1), Tumor Necrosis Factor Receptor 2 (TNFR2), Interleukin-2 Simoa (IL-2 Simoa), Monokine Induced by Gamma Interferon (MIG; CXCL9), EN-RAGE, Tumor Necrosis Factor Receptor 1 (TNF R1), Eotaxin-2, Chemokine CC-4 (HCC-4), Urokinase-type Plasminogen Activator Receptor (uPAR), Interleukin-2 Receptor Alpha (IL-2 receptor alpha), Macrophage Inflammatory Protein-1 beta (MIP-1 beta), Interferon gamma Induced Protein 10 (IP-10; CXCL10), 6Ckine, Macrophage Inflammatory Protein-3 beta (MIP-3 beta), Macrophage-Derived Chemokine (MDC), AXL Receptor Tyrosine Kinase (AXL), and Tissue Inhibitor of Metalloproteinases 1 (TIMP-1). In some embodiments, an increase in the level of one or more members of the panel correlates with an increased likelihood of a positive clinical outcome in a patient, or indicates that the patient should continue treatment. In some embodiments, an increase in the level of one or more members of the panel correlates with an increased likelihood of a negative clinical outcome in a patient, or indicates that the patient should not continue treatment.

In some embodiments, the biomarkers of a cytokine panel comprise one or more of Plasminogen Activator Inhibitor 1 (PAI-1), Brain-Derived Neurotrophic Factor (BDNF), Epidermal Growth Factor (EGF), E-Selectin, and Monocyte Chemotactic Protein 2 (MCP-2). In some embodiments, a decrease in the level of one or more members of the panel correlates with an increased likelihood of a positive clinical outcome in a patient, or indicates that the patient should continue treatment. In some embodiments, a decrease in the level of one or more members of the panel correlates with an increased likelihood of a negative clinical outcome in a patient, or indicates that the patient should not continue treatment.

As referred to herein, a “cytokine gene signature” or “cytokine signature” refers to cytokine related genes. In some embodiments, the cytokine signature is selected from a change (i.e., an increase or decrease) of one or more of IL6, IL7, CXCL8, IL10, IL12A, IL22, IL23a, IFNA2, IFNG, LTA, CCL2, CXCL12, CXCL10, CXCL9, CSF2, PDGFB, HGF, and VEGFA. In some embodiments, the cytokine gene signature is selected from a change (i.e., an increase or decrease) of two or more of IL6, IL7, CXCL8, IL10, IL12A, IL22, IL23a, IFNA2, IFNG, LTA, CCL2, CXCL12, CXCL10, CXCL9, CSF2, PDGFB, HGF, and VEGFA. In some embodiments, the cytokine gene signature is selected from a change (i.e., an increase or decrease) of three or more of IL6, IL7, CXCL8, IL10, IL12A, IL22, IL23a, IFNA2, IFNG, LTA, CCL2, CXCL12, CXCL10, CXCL9, CSF2, PDGFB, HGF, and VEGFA. In some embodiments, the cytokine gene signature is selected from a change (i.e., an increase or decrease) of four or more of IL6, IL7, CXCL8, IL10, IL12A, IL22, IL23a, IFNA2, IFNG, LTA, CCL2, CXCL12, CXCL10, CXCL9, CSF2, PDGFB, HGF, and VEGFA. In some embodiments, the cytokine gene signature is selected from a change (i.e., an increase or decrease) of five or more of IL6, IL7, CXCL8, IL10, IL12A, IL22, IL23a, IFNA2, IFNG, LTA, CCL2, CXCL12, CXCL10, CXCL9, CSF2, PDGFB, HGF, and VEGFA. In some embodiments, the cytokine gene signature is selected from a change (i.e., an increase or decrease) often or more of IL6, IL7, CXCL8, IL10, IL12A, IL22, IL23a, IFNA2, IFNG, LTA, CCL2, CXCL12, CXCL10, CXCL9, CSF2, PDGFB, HGF, and VEGFA. In some embodiments, the cytokine gene signature is selected from a change (i.e. an increase or decrease) of fifteen or more of IL6, IL7, CXCL8, IL10, IL12A, IL22, IL23a, IFNA2, IFNG, LTA, CCL2, CXCL12, CXCL10, CXCL9, CSF2, PDGFB, HGF, and VEGFA. In some embodiments, the cytokine gene signature is selected from a change (i.e., an increase or decrease) of all of IL6, IL7, CXCL8, IL10, IL12A, IL22, IL23a, IFNA2, IFNG, LTA, CCL2, CXCL12, CXCL10, CXCL9, CSF2, PDGFB, HGF, and VEGFA.

In some embodiments, the cytokine signature is selected from a change (i.e., an increase or decrease) of one or more of TNFRSF10C, IL6R, CCL23, TNFRSF1B, IL2, CXCL9, S100A12, TNFRSF1A, CCR3, CCL16, PLAUR, IL2RA, CCL4, CXCL10, CCL21, CCL19, CCL22, AXL, TIMP1, SERPINE1, BDNF, EGF, SELE, and CCL8, or a net increase or decrease of the group as a whole, in a serum sample relative to a control. In some embodiments, an increase or decrease in one, two, three, four, five, ten, fifteen, twenty, or all of TNFRSF10C, IL6R, CCL23, TNFRSF1B, IL2, CXCL9, S100A12, TNFRSF1A, CCR3, CCL16, PLAUR, IL2RA, CCL4, CXCL10, CCL21, CCL19, CCL22, AXL, TIMP1, SERPINE1, BDNF, EGF, SELE, and CCL8 in a serum sample from a patient who has undergone treatment with X4P-001 correlates with an increased likelihood that the patient will benefit from continued treatment with X4P-001 alone or in combination with an immunotherapeutic agent, e.g., a checkpoint inhibitor such as a PD-1 antagonist.

It has been surprisingly found that X4P-001 increases one or more of a panel of IFN-γ related genes referred to herein as an “IFN-γ gene signature.” In some embodiments, the IFN-γ gene signature is selected from a change (i.e., an increase or decrease) of one or more of IDO1, CXCL10, CXCL9, HLA-DRA, STAT1 and IFN-γ, or a net increase or decrease of the group as a whole, in a tumor relative to a control. In some embodiments, the biomarker is IDO1. In some embodiments, the biomarker is CXCL10. In some embodiments, the biomarker is CXCL9. In some embodiments, the biomarker is HLA-DRA. In some embodiments, the biomarker is STAT1. In some embodiments, the biomarker is IFN-γ. In some embodiments, the biomarker is two or more of IDO1, CXCL10, CXCL9, HLA-DRA, STAT1, and IFN-γ. In some embodiments, the biomarker is three or more of IDO1, CXCL10, CXCL9, HLA-DRA, STAT1 and IFN-γ. In some embodiments, the biomarker is four or more of IDOL, CXCL10, CXCL9, HLA-DRA, STAT1, and IFN-γ. In some embodiments, the biomarker is five or more of IDO1, CXCL10, CXCL9, HLA-DRA, STAT1, and IFN-γ. In some embodiments, the biomarker is all of IDO1, CXCL10, CXCL9, HLA-DRA, STAT1, and IFN-γ. In some embodiments, an increase or decrease in one, two, three, four, five, or all of IDO1, CXCL10, CXCL9, HLA-DRA, STAT1, and IFN-γ in a tumor sample from a patient who has undergone treatment with X4P-001 correlates with an increased likelihood that the patient will benefit from continued treatment with X4P-001 alone or in combination with an immunotherapeutic agent, e.g., a checkpoint inhibitor such as a PD-1 antagonist. In some embodiments, the PD-1 antagonist is selected from nivolumab, pembrolizumab, a pembrolizumab biosimilar, or a pembrolizumab variant. In some embodiments, the checkpoint inhibitor is pembrolizumab. In some embodiments, the tumor is a solid tumor such as advanced or metastatic melanoma. In some embodiments, the biomarker or the use thereof is one of those described in Ayers et al., Journal of Clinical Investigation 2017, 127(8), 2930-2940 [29] (“Ayers et al. (2017)”) or WO 2016/094377, each of which is hereby incorporated by reference.

In other embodiments the biomarker is two, three, four, five, six, seven, eight, about ten, about twenty, or more of an expanded 28-gene immune signature consisting of: IL2Rg; CXCR6; CD3d; CD2; ITGAL; TAGAP; CIITA; HLA-DRA; PTPRC; CXCL9; CCL5; NKG7; GZMA; PRF1; CCR5; CD3e; GZMK; IFNG; HLA-E; GZMB; PDCD1; SLAMF6; CXCL13; CXCL10; IDO1; LAGS; STAT1; and CXCL11; or an expanded 10-gene IFN-γ signature comprising IFNG, STAT1, CCR5, CXCL9, CXCL10, CXCL11, IDO1, PRF1, GZMA, and MHCII HLA-DRA. Ayers et al. (2017).

In some embodiments, a cytokine signature comprises a decrease in expression of one or more of ANG-1, ENA-78, Latency-associated peptide of transforming growth factor beta 1, MCP-1 and PDGF-BB after administration of a CXCR4 inhibitor.

In some embodiments, a cytokine signature comprises an increase in expression of one or more of 6CKine, Decorin, IL-2, MIP-3 beta, MIG (CXCL9) and MPIF-1P after administration of a CXCR4 inhibitor.

In other embodiments the biomarker is one or more of a panel of antigen presentation/procession related genes referred to herein as an “antigen presentation/processing gene signature.” In some embodiments, the antigen presentation/processing gene signature is selected from a change (i.e., an increase or decrease) of one or more of B2M, CD74, CTSL, CTSS, HLA-DMA, HLA-DMB, HLA-DOB, HLA DPA1, HLA-DPB1, HLA-DQA1, HLA-DQB1, HLA-DRA, HLA-DRB1, HLA-DRB3, PSMB8, PSMB9, TAP1, and TAP2, or a net increase or decrease of the group as a whole, in a tumor relative to a control. In some embodiments, an increase or decrease in one, two, three, four, five, ten, fifteen, or all of B2M, CD74, CTSL, CTSS, HLA-DMA, HLA-DMB, HLA-DOB, HLA DPA1, HLA-DPB1, HLA-DQA1, HLA-DQB1, HLA-DRA, HLA-DRB1, HLA-DRB3, PSMB8, PSMB9, TAP1, and TAP2 in a tumor sample from a patient who has undergone treatment with X4P-001 correlates with an increased likelihood that the patient will benefit from continued treatment with X4P-001 alone or in combination with an immunotherapeutic agent, e.g., a checkpoint inhibitor such as a PD-1 antagonist.

In other embodiments the biomarker is one or more of a panel of tumor inflammation related genes referred to herein as a “tumor inflammation gene signature.” In some embodiments, the tumor inflammation gene signature is selected from a change (i.e., an increase or decrease) of one or more of CCL5, CD27, CD274, CD276, CD8A, CMKLR1, CXCL9, CXCR6, HLA-DQA1, HLA-DRB1, HLA-E, IDOL, LAG3, NKG7, PDCD1LG2, PSMB10, STAT1, and TIGIT, or a net increase or decrease of the group as a whole, in a tumor relative to a control. In some embodiments, an increase or decrease in one, two, three, four, five, ten, fifteen, or all of CCL5, CD27, CD274, CD276, CD8A, CMKLR1, CXCL9, CXCR6, HLA-DQA1, HLA-DRB1, HLA-E, IDOL, LAG3, NKG7, PDCD1LG2, PSMB10, STAT1, and TIGIT in a tumor sample from a patient who has undergone treatment with X4P-001 correlates with an increased likelihood that the patient will benefit from continued treatment with X4P-001 alone or in combination with an immunotherapeutic agent, e.g., a checkpoint inhibitor such as a PD-1 antagonist.

It has surprisingly been found that X4P-001 treats cancers such as solid tumors, e.g., advanced or metastatic melanoma, without significantly increasing levels of T_{reg} cells. Without wishing to be bound by theory, it is believed that because T_{reg} cells inhibit immune response, this indicates that the tumor microenvironment is exhibiting a significant increase in this immune regulatory response that would normally allow the tumor to evade host immunity. Accordingly, in some embodiments, the biomarker is maintenance or decrease of T_{reg} levels in a tumor relative to a control. In some embodiments, the biomarker is the level of FoxP3 expression, which serves as a means to determine the T_{reg} level. In some embodiments, the biomarker is an increase in the ratio of CD8⁺ T cells/FoxP3 in the tumor microenvironment or tumor sample. In some embodiments, the measured increase of the biomarker in a tumor sample from a patient who has undergone treatment with X4P-001 correlates with an increased likelihood that the patient will benefit from continued treatment with X4P-001 alone or in combination with an immunotherapeutic agent, e.g., a checkpoint inhibitor such as a PD-1 antagonist. In some embodiments, the PD-1 antagonist is selected from nivolumab and pembrolizumab, or a biosimilar or variant of such PD-1 antagonists. In some embodiments, the checkpoint inhibitor is nivolumab. In some embodiments, the checkpoint inhibitor is a nivolumab biosimilar or variant. In some embodiments, the checkpoint inhibitor is pembrolizumab. In some embodiments, the checkpoint inhibitor is a pembrolizumab biosimilar or variant. In some embodiments, the tumor is a solid tumor such as advanced or metastatic melanoma.

It has surprisingly been found that X4P-001 treats cancers such as solid tumors, e.g., advanced or metastatic melanoma, without significantly modulating levels of macrophages in the tumor. Accordingly, in some embodiments, the biomarker is maintenance or approximate maintenance of macrophage levels in the tumor relative to a control.

It has surprisingly been found that X4P-001 increases PD-L1 expression in tumor samples and the tumor microenvironment. Without wishing to be bound by theory, it has been proposed that PD-L1 expressing tumor cells interact with PD-1 expressing T cells to attenuate T cell activation and evasion of immune surveillance, thereby contributing to an impaired immune response against the tumor. Accordingly, in some embodiments, the biomarker is an increase in PD-L1 expression. In some embodiments, increase of the biomarker in a tumor sample from a patient who has undergone treatment with X4P-001 correlates with an increased likelihood that the patient will benefit from continued treatment with X4P-001 alone or in combination with an immunotherapeutic agent, e.g., a checkpoint inhibitor such as a PD-1 antagonist. In some embodiments, the PD-1 antagonist is selected from nivolumab and pembrolizumab, or a biosimilar or variant of such PD-1 antagonists. In some embodiments, the checkpoint inhibitor is nivolumab. In some embodiments, the checkpoint inhibitor is a nivolumab biosimilar or variant. In some embodiments, the checkpoint inhibitor is pembrolizumab. In some embodiments, the checkpoint inhibitor is a pembrolizumab biosimilar or variant. In some embodiments, the tumor is a solid tumor such as advanced or metastatic melanoma.

It has surprisingly been found that X4P-001 increases gene expression of one or more of a panel of cytotoxic T cell (CTL)-related genes referred to herein as a “CTL signature” in tumor samples or the tumor microenvironment. Accordingly, in some embodiments, the biomarker is an increase in the CTL signature. In some embodiments, the CTL signature comprises an increase in one or more of CD8A, CD8B, FLTLG, GZMM, or PRF1. In some embodiments, the CTL signature comprises an increase in two or more, three or more, four or more, or each of CD8A, CD8B, FLTLG, GZMM, or PRF1. In some embodiments, the biomarker is a net increase in total expression of the CTL signature. In some embodiments, increase of the biomarker in a tumor sample from a patient who has undergone treatment with X4P-001 correlates with an increased likelihood that the patient will benefit from continued treatment with X4P-001 alone or in combination with an immunotherapeutic agent, e.g., a checkpoint inhibitor such as a PD-1 antagonist. In some embodiments, the PD-1 antagonist is selected from nivolumab and pembrolizumab, or a biosimilar or variant of such PD-1 antagonists. In some embodiments, the checkpoint inhibitor is nivolumab. In some embodiments, the checkpoint inhibitor is a nivolumab biosimilar or variant. In some embodiments, the checkpoint inhibitor is pembrolizumab. In some embodiments, the checkpoint inhibitor is a pembrolizumab biosimilar or variant. In some embodiments, the tumor is a solid tumor such as advanced or metastatic melanoma.

In accordance with the present invention, biomarkers may be measured before, during, and/or after treatment with a CXCR4 inhibitor and, optionally, an immunotherapeutic agent, and then correlated with clinical outcomes, response rates, prognoses, or another predictive or interpretative measurement.

The system and methods of the present invention are based in part on a combination of a clinical response biomarker (e.g., gene) set and a normalization biomarker (e.g., gene) set, referred to herein as a “biomarker expression platform,” which is employed as a tool for deriving different sets of genes having pre-treatment intratumoral biomarker, e.g., RNA expression, levels (“biomarker signatures” or “gene signatures”) that are correlated with an anti-tumor response to a CXCR4 inhibitor optionally in combination with a PD-1 antagonist for multiple tumor types. This biomarker expression platform is useful to derive a scoring algorithm that weights the relative contribution of individual biomarkers in a signature to a correlation to generate an arithmetic composite of normalized biomarker levels of all of the biomarkers, such as genes in the gene signature, referred to herein as a “gene signature score.” By comparing gene signature scores and anti-tumor responses obtained for a cohort of patients with the same tumor type of interest and treated with a CXCR4 inhibitor optionally in combination with a PD-1 antagonist, a cut-off score may be selected that divides patients according to having a higher or lower probability of achieving an anti-tumor response to treatment. A predictive signature score for a particular tumor type is referred to herein as a gene signature biomarker. Patients whose tumors test positive for a biomarker signature or gene signature biomarker derived according to the present invention are more likely to benefit from therapy with a CXCR4 inhibitor optionally in combination with a PD-1 antagonist than patients whose tumors test negative for the biomarker signature or gene signature biomarker.

Thus, in a first aspect, the invention provides a method of deriving a gene signature biomarker that is predictive of an anti-tumor response to a CXCR4 inhibitor optionally in combination with a PD-1 antagonist for at least one tumor type of interest. The method comprises: (a) obtaining a pre-treatment tumor sample from each patient in a patient cohort diagnosed with the tumor type; (b) obtaining, for each patient in the cohort, an anti-tumor response value following treatment with the CXCR4 inhibitor optionally in combination with a PD-1 antagonist; (c) measuring the raw RNA levels in each tumor sample for each gene in a gene expression platform, wherein the gene expression platform comprises a set of clinical response genes and a set of normalization genes; (d) normalizing, for each tumor sample, each of the measured raw RNA levels for the clinical response genes using the measured RNA levels of the normalization genes; (e) optionally weighting, for each tumor sample and each gene in a gene signature of interest, the normalized RNA expression levels using a pre-defined multiplication coefficient for that gene; (f) optionally adding, for each tumor sample, the weighted RNA expression levels to generate a gene signature score; and (g) comparing the normalized RNA levels or gene signature scores for all of the tumor samples and anti-tumor response values for all of the patients in the cohort to select a cut-off for the RNA levels or gene signature score, respectively, that divides the patient cohort to meet a target biomarker clinical utility criterion. In an embodiment, the method further comprises designating any tumor sample of the tumor type that has a gene signature score that is equal to or greater than the selected cut-off as biomarker high and designating any tumor sample of the tumor type that has a gene signature score that is below the selected cutoff as biomarker low.

The inventors contemplate that gene signature biomarkers derived using the above method of the invention would be useful in a variety of clinical research and patient treatment settings, such as, for example, to selectively enroll only biomarker high patients into a clinical trial of a CXCR4 inhibitor optionally in combination with a PD-1 antagonist, to stratify the analysis of a clinical trial of a CXCR4 inhibitor optionally in combination with a PD-1 antagonist based on biomarker high or negative status, or to determine eligibility of a patient for treatment with a CXCR4 inhibitor optionally in combination with a PD-1 antagonist.

Thus, in a second aspect, the invention provides a method for testing a tumor sample removed from a patient diagnosed with a particular tumor type for the presence or absence of a gene signature biomarker of anti-tumor response of the tumor type to a CXCR4 inhibitor optionally in combination with a PD-1 antagonist. The method comprises: (a) measuring the raw RNA level in the tumor sample for each gene in a gene expression platform, wherein the gene expression platform comprises a set of clinical response genes and a set of normalization genes; (b) normalizing the measured raw RNA level for each clinical response gene in a pre-defined gene signature for the tumor type using the measured RNA levels of the normalization genes; (c) optionally weighting each normalized RNA value using a pre-defined multiplication co-efficient; (d) optionally adding the weighted RNA expression levels to generate a gene signature score; (e) comparing the normalized RNA level or generated score to a reference score or reference RNA level for the gene signature and tumor type; and (f) classifying the tumor sample as biomarker high or biomarker low; wherein if the generated score is equal to or greater than the reference score or measured RNA level is greater than the reference RNA level, then the tumor sample is classified as biomarker high, and if the generated score is less than the reference score or measured RNA level is less than the reference RNA level, then the tumor sample is classified as biomarker low.

In a third aspect, the invention provides a system for testing a tumor sample removed from a patient diagnosed with a particular tumor type for the presence or absence of a gene signature biomarker of anti-tumor response of the tumor type to a CXCR4 inhibitor optionally in combination with a PD-1 antagonist. The system comprises (i) a sample analyzer for measuring raw RNA expression levels of each gene in a gene expression platform, wherein the gene expression platform consists of a set of clinical response genes and a set of normalization genes, and (ii) a computer program for receiving and analyzing the measured RNA expression levels to (a) normalize the measured raw RNA level for each clinical response gene in a pre-defined gene signature for the tumor type using the measured RNA levels of the normalization genes; (b) optionally weight each normalized RNA value using a pre-defined multiplication co-efficient; (c) optionally add the weighted RNA expression levels to generate a gene signature score; (d) compare the normalized RNA levels or generated score to reference RNA levels or a reference score for the gene signature and tumor type; and (e) classify the tumor sample as biomarker high or biomarker low, wherein if the generated score is equal to or greater than the reference score or normalized RNA levels are greater than the reference levels, then the tumor sample is classified as biomarker high, and if the generated score is less than the reference score or normalized RNA levels are less than the reference levels, then the tumor sample is classified as biomarker low.

In each of the above aspects of the invention, the clinical response genes in the gene expression platform are (a) individually correlated with an anti-tumor response to normalized RNA levels in more than one tumor type and (b) collectively generate a covariance pattern that is substantially similar in each of the tumor types. A first subset of genes in the clinical response gene set exhibit intratumoral RNA levels that are positively correlated with the antitumor response while intratumoral RNA levels for a second subset of genes in the clinical response gene set are negatively correlated with the anti-tumor response. In an embodiment, the clinical response gene set comprises about 2-25 genes.

In some embodiments of any of the above aspects of the invention, the set of normalization genes in the gene expression platform comprises genes which individually exhibit intratumoral RNA levels of low variance across multiple samples of the different tumor types and collectively exhibit a range of intratumoral RNA levels that spans the range of intratumoral expression levels of the clinical response genes in the different tumor types. In some embodiments, the normalization gene set comprises about 10 to 12 genes.

In some embodiments, the biomarker or gene signature or normalization gene set is one of those disclosed in WO 2016/094377, the disclosure of which is hereby incorporated by reference.

### Dosage and Formulation

X4P-001 is a CXCR4 antagonist with molecular formula C₂₁H₂₇N₅; molecular weight 349.48 amu; appearance: white to pale yellow solid; solubility: freely soluble in the pH range 3.0 to 8.0 (>100 mg/mL), sparingly soluble at pH 9.0 (10.7 mg/mL) and slightly soluble at pH 10.0 (2.0 mg/mL). X4P-001 is only slightly soluble in water; and has a melting point of 108.9° C.

In certain embodiments, the composition containing X4P-001 is administered orally, in an amount from about 200 mg to about 1200 mg daily. In certain embodiments, the dosage composition may be provided twice a day in divided dosage, approximately 12 hours apart. In other embodiments, the dosage composition may be provided once daily. The terminal half-life (T1/2) of X4P-001 has been generally determined to be between about 12 to about 24 hours, or approximately 14.5 hours. Dosage for oral administration may be from about 100 mg to about 1200 mg once or twice per day. In certain embodiments, the dosage of X4P-001 useful in the invention is from about 200 mg to about 600 mg daily. In other embodiments, the dosage of X4P-001 useful in the invention may range from about 400 mg to about 800 mg, from about 600 mg to about 1000 mg or from about 800 mg to about 1200 mg daily. In certain embodiments, the invention comprises administration of an amount of X4P-001 of about 10 mg, about 20 mg, about 25 mg, about 50 mg, about 75 mg, about 100 mg, about 125 mg, about 150 mg, about 200 mg, about 250 mg, about 300 mg, about 400 mg, about 450 mg, about 500 mg, about 600 mg, about 650 mg, about 700 mg, about 750 mg, about 800 mg, about 850 mg, about 900 mg, about 950 mg, about 1000 mg, about 1100 mg, about 1200 mg, about 1300 mg, about 1400 mg, about 1500 mg or about 1600 mg.

In some embodiments, a provided method comprises administering to the patient a pharmaceutically acceptable composition comprising X4P-001 wherein the composition is formulated for oral administration. In certain embodiments, the composition is formulated for oral administration in the form of a tablet or a capsule. In some embodiments, the composition comprising X4P-001 is formulated for oral administration in the form of a capsule.

In certain embodiments, a provided method comprises administering to the patient one or more capsules comprising 100-1200 mg X4P-001 active ingredient; and one or more pharmaceutically acceptable excipients.

In certain embodiments, the present invention provides a composition comprising X4P-001, or a pharmaceutically acceptable salt thereof, one or more diluents, a disintegrant, a lubricant, a flow aid, and a wetting agent. In some embodiments, the present invention provides a composition comprising 10-1200 mg X4P-001, or a pharmaceutically acceptable salt thereof, microcrystalline cellulose, dibasic calcium phosphate dihydrate, croscarmellose sodium, sodium stearyl fumarate, colloidal silicon dioxide, and sodium lauryl sulfate. In some embodiments, the present invention provides a unit dosage form wherein said unit dosage form comprises a composition comprising 10-200 mg X4P-001, or a pharmaceutically acceptable salt thereof, microcrystalline cellulose, dibasic calcium phosphate dihydrate, croscarmellose sodium, sodium stearyl fumarate, colloidal silicon dioxide, and sodium lauryl sulfate. In certain embodiments, the present invention provides a unit dosage form comprising a composition comprising X4P-001, or a pharmaceutically acceptable salt thereof, present in an amount of about 10 mg, about 20 mg, about 25 mg, about 50 mg, about 75 mg, about 100 mg, about 125 mg, about 150 mg, about 200 mg, about 250 mg, about 300 mg, about 400 mg, about 450 mg, about 500 mg, about 600 mg, about 650 mg, about 700 mg, about 750 mg, about 800 mg, about 850 mg, about 900 mg, about 950 mg, about 1000 mg, about 1100 mg, about 1200 mg, about 1300 mg, about 1400 mg, about 1500 mg or about 1600 mg. In some embodiments, a provided composition (or unit dosage form) is administered to the patient once per day, twice per day, three times per day, or four times per day. In some embodiments, a provided composition (or unit dosage form) is administered to the patient once per day or twice per day.

In some embodiments, the present invention provides a unit dosage form comprising a composition comprising:
(a) X4P-001, or a pharmaceutically acceptable salt thereof in about 30-40% by weight of the composition;
(b) microcrystalline cellulose in about 20-25% by weight of the composition;
(c) dibasic calcium phosphate dihydrate in about 30-35% by weight of the composition;
(d) croscarmellose sodium in about 5-10% by weight of the composition;
(e) sodium stearyl fumarate in about 0.5-2% by weight of the composition;
(f) colloidal silicon dioxide in about 0.1-1% by weight of the composition; and
(g) sodium lauryl sulfate in about 0.1-1.0% by weight of the composition.

In some embodiments, the present invention provides a unit dosage form comprising a composition comprising:
(a) X4P-001, or a pharmaceutically acceptable salt thereof—about 37% by weight of the composition;
(b) microcrystalline cellulose in about 23% by weight of the composition;
(c) dibasic calcium phosphate dihydrate in about 32% by weight of the composition;
(d) croscarmellose sodium in about 6% by weight of the composition;
(e) sodium stearyl fumarate in about 1% by weight of the composition;
(f) colloidal silicon dioxide in about 0.3% by weight of the composition; and
(g) sodium lauryl sulfate in about 0.5% by weight of the composition.

In some embodiments, the present invention provides a unit dosage form comprising a composition comprising:
(a) X4P-001, or a pharmaceutically acceptable salt thereof in about 55-65% by weight of the composition;
(b) microcrystalline cellulose in about 10-15% by weight of the composition;
(c) dibasic calcium phosphate dihydrate in about 15-20% by weight of the composition;
(d) croscarmellose sodium in about 5-10% by weight of the composition;
(e) sodium stearyl fumarate in about 0.5-2% by weight of the composition;
(f) colloidal silicon dioxide in about 0.1-1.0% by weight of the composition; and
(g) sodium lauryl sulfate in about 0.1-1.0% by weight of the composition.

Pembrolizumab has been approved by the FDA for treatment of unresectable or metastatic melanoma or metastatic non-small cell lung cancer, and is generally administered at a dosage of 2 mg/kg as an intravenous infusion over 30 minutes once every 3 weeks. Generally, the amount of pembrolizumab or other immune checkpoint inhibitor useful in the present invention will be dependent upon the size, weight, age and condition of the patient being treated, the severity of the disorder or condition, and the discretion of the prescribing physician.

Inasmuch as it may be desirable to administer a combination of active compounds, for example, for the purpose of treating a particular disease or condition, it is within the scope of the present invention that two or more pharmaceutical compositions, at least one of which contains a compound in accordance with the invention, may conveniently be combined in the form of a kit suitable for co-administration of the compositions. Thus, in some embodiments, the invention provides a kit that includes two or more separate pharmaceutical compositions, at least one of which contains a compound of the invention, and means for separately retaining said compositions, such as a container, divided bottle, or divided foil packet. An example of such a kit is the familiar blister pack used for the packaging of tablets, capsules and the like.

The kit of the invention is particularly suitable for administering different dosage forms, for example, oral and parenteral, for administering the separate compositions at different dosage intervals, or for titrating the separate compositions against one another. To assist compliance, the kit typically includes directions for administration and may be provided with a memory aid.

The examples below explain the invention in more detail. The following preparations and examples are given to enable those skilled in the art to more clearly understand and to practice the present invention. The present invention, however, is not limited in scope by the exemplified embodiments, which are intended as illustrations of single aspects of the invention only, and methods which are functionally equivalent are within the scope of the invention. Indeed, various modifications of the invention in addition to those described herein will become apparent to those skilled in the art from the foregoing description and accompanying drawings. Such modifications are intended to fall within the scope of the appended claims.

The contents of each document cited in the specification are herein incorporated by reference in their entireties.

### EXEMPLIFICATION

### Example 1: Nine Week Monotherapy and Combination Therapy Study in Patients with Malignant Melanoma with Measurement of Biomarkers

Treatment with X4P-001 as a monotherapy, or in combination with a checkpoint inhibitor, such as pembrolizumab, may be performed in cycles, such as on a 3 week or 9 week cycle. In certain embodiments, the cycle is 9 weeks long. X4P-001 at a determined dose from 200 mg to 1200 mg daily is administered orally either once daily or twice daily in divided doses. Patients are instructed about both dosing schedule and requirements relating to food or drink near the time of dosing.

Dosing Schedule. The daily dose is taken first thing in the morning. Where the dose is divided, the first daily dose is taken in the morning and the second daily dose approximately 12 hours later using the following guidelines:
Dosing should be at the same time(s) each day±2 hr.
For twice daily dosing, the interval between successive doses should not be <9 hours nor >15 hours. If the interval would be >15 hrs, the dose should be omitted and the usual schedule resumed at the next dose.
Restrictions relating to food. Absorption is impacted by food and patients will be instructed as follows:

For the Morning Dose
No food or drink (except water) after midnight until the time of dosing
No food or drink (except water) for 2 hour after dosing.

For the Second Daily Dose, if Applicable
No food or drink (except water) for 1 hour before dosing
No food or drink (except water) for 2 hours after dosing.

Pembrolizumab is administered consistent with prescribed labeling information. Concomitant treatment with X4P-001 and pembrolizumab may be administered, beginning with daily administration of X4P-001 at day 1. Initial treatment with pembrolizumab is at 2 mg/kg administered by intravenous infusion over 30 minutes in clinic at the week 4 and 7 visits. Patients may, with the approval of their clinician, vary the dosing schedule or dosage of pembrolizumab.

Dosing of X4P-001 and/or pembrolizumab may be adjusted by the clinician as appropriate. The dose of X4P-001 and/or pembrolizumab may be lowered according to the judgment of the clinician. If a patient receiving X4P-001 in combination with pembrolizumab experiences an adverse event at Grade>2, the dose of X4P-001 and/or pembrolizumab may be lowered according to the judgment of the clinician. If a patient successfully completes the first 4 weeks of treatment, that is, without experiencing any adverse events greater than Grade 2, the daily dose of X4P-001 and/or pembrolizumab may be increased, consistent with the judgment of the clinician.

Patients with resectable metastatic melanoma, after combination treatment with X4P-001 and pembrolizumab, will typically undergo complete resection, or resection that is as complete as possible, and could continue to be monitored for recurrence, and/or undergo standard of care (SOC) treatment. This could mean continued use of pembrolizumab, or it could mean some other treatment at the clinician's discretion. Patients with unresectable metastatic melanoma, after treatment, will continue to undergo SOC treatment. Such SOC treatment may or may not include a further regimen of X4P-001, with or without pembrolizumab.

### Evaluation of Response to Treatment and Disease Status

Baseline radiologic assessment of the patient is conducted in order to confirm whether the patient has resectable disease. At end of treatment, repeat imaging will be performed using the same modality.

At initial assessment, the patient is diagnosed as having malignant melanoma, including Stage III (any substage) or Stage IV (with isolated skin metastasis only). Patient is assessed for cutaneous/subcutaneous lesions, including those that will be biopsied clinically.

Cutaneous/subcutaneous lesions≥3 mm are assessed clinically by the investigator, including the number, distribution, and a description of the lesions (e.g., nodular, popular, macular, pigmented, etc.). The size of the cutaneous lesions is determined using photographs of the lesions (including a ruler with patient study identification and date) obtained as indicated in the schedule of events. Lymph nodes are examined at each visit and the location and size of palpable nodes recorded.

Clinical assessments of cutaneous/subcutaneous disease are conducted at each of day 1, week 4 and week 7, and as indicated based on new signs, symptoms or laboratory findings. Assessments will include physical examination (including lymph nodes) and photographs of all cutaneous lesions, including a ruler marked with patient study number and date.

### Biomarker Assessments

If desired, pharmacokinetic assessment of blood samples for plasma levels of X4P-001 and pembrolizumab may be conducted. Blood samples are collected as scheduled. For example, samples may be taken at day 1, week 4 and week 7. Samples are analyzed for X4P-001 concentration using reversed-phase high performance liquid chromatography (RP-HPLC) with MS/MS detection. The validated range of this bioanalytic method is 30 to 3,000 ng/mL in plasma.

The initial measurement at day 1 is designated as baseline. At week 4 and week 7, measurements of CD8⁺ T cells are taken and compared to baseline.

A primary comparison is the density of specific cell phenotypes in the tumor microenvironment in the pre-treatment biopsy vs. the Week 4 and EOT biopsies. CD8⁺ T cells/mm⁻² are measured in melanoma tumor parenchyma prior to treatment.

An increase at week 4 compared to baseline is considered to be a positive response.

Secondary analyses include (a) comparison of cell phenotypes in the Week 4 vs. EOT biopsies, (b) changes over time in phenotypes among peripheral blood mononuclear cells (PBMCs and in serum biomarker levels. Normally distributed continuous variables are analyzed using t-test and ANOVA/ANCOVA, as appropriate. Variables whose results are not normally distributed are analyzed by non-parametric statistics. Fisher's exact test is used for categorical variables.

Pharmacokinetic assessment of pembrolizumab may be accomplished using techniques, such as those described in Patnaik et al. (2015) Clin. Cancer Res. 21:4286-4293, the full disclosure of which is hereby specifically incorporated herein by reference.

### Example 2: Nine Week Monotherapy and Combination Therapy Study in Patients with Malignant Melanoma with Measurement of Biomarkers

### Clinical Protocol

A total of sixteen (16) patients were enrolled in a controlled study. The study population was comprised of male and female adult subjects (≥18 years of age) with histologically confirmed malignant melanoma. Subjects were further required to have at least two (2) separate cutaneous or subcutaneous lesions suitable for punch biopsies (≥3 mm).

Subjects were excluded if they had an Eastern Cooperative Oncology Group (ECOG) performance score of two (2) or greater. Subjects were further excluded is they had previously received checkpoint inhibitor therapies (e.g., anti-CTLA-4, PD-1, PD-L1) or oncolytic virus therapy. Subjects with ongoing HIV, hepatitis C, or uncontrollable infections were excluded, as were subjects who had myocardial infarctions, grade three (3) or higher hemorrhage, chronic liver disease, or other active malignancies within the previous six (6) months.

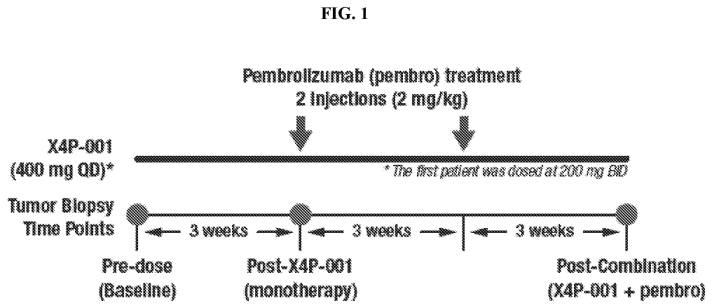

Subjects were first screened and evaluated for baseline measurements. Enrolled participants received treatment a cycle involving a first period comprising X4P-001 monotherapy and a second period comprising of X4P-001 and a checkpoint inhibitor combination therapy. The dosing schedule for the study is summarized in FIG. 1.

Prior to treatment two (2) baseline serum samples were collected from each patient. One baseline serum sample was collected at the time of screening and another was collected one to four weeks later on Day 1 of the treatment, prior to the administration of the first dose of X4P-001. In addition to the baseline serum samples, a baseline punch biopsy was collected from each patient on D1 prior to the administration of X4P-001.

Beginning on Day 1 subjects received 400 mg of X4P-001 orally, q.i.d. One patient received 200 mg orally, b.i.d. Patients were administered X4P-001 throughout the nine (9) week study.

Three (3) weeks after treatment was initiated, additional serum samples were collected from each patient. Additional biopsy samples were also collected unless the attending physician recommended against the biopsy. Following sample collection, subjects were administered the first of two doses of pembrolizumab (2 mg/kg, i.v.).

Three (3) weeks after the administration of the first dose of pembrolizumab (six weeks from beginning of treatment) additional serum samples were collected from each patient. Subjects then administered a second dose of pemrolizumab (2 mg/kg, i.v.).

Three (3) weeks after the administration of the second dose of pembrolizumab (nine weeks from the beginning of treatment) additional serum samples were collected. Additional biopsy samples were also collected unless the attending physician recommended against the biopsy.

### Serum Biomarkers Investigation

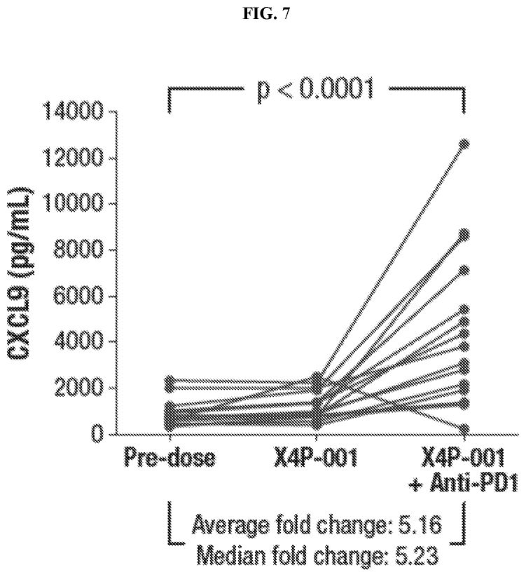

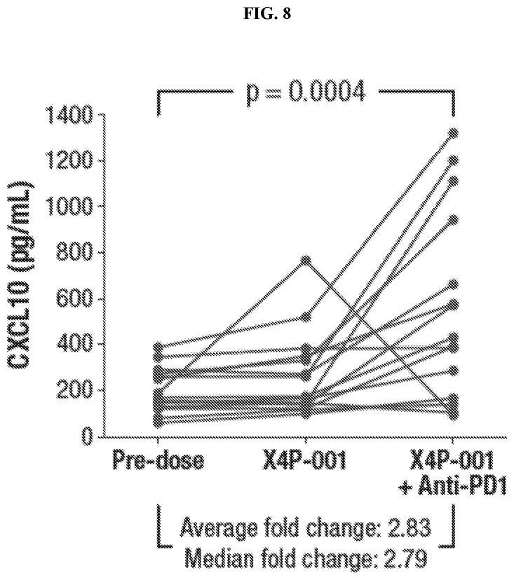

Blood samples from melanoma patients were collected at screening, D1, D21 (three weeks single agent therapy), D42 (three weeks combination treatment), (D63 (six weeks combination treatment). Chemokines, cytokines, and growth factors in serum were measured using the Multi-Analyte Profile Platform (Myriad RBM). Serum concentrations of CXCL9 and CXCL10 at baseline, following monotherapy with X4P-001, and following combination treatment with X4P-001 and pembro for eleven (11) study subjects are shown in Table 1, Table 1a, Table 2, and Table 2a, below. Results show that CXCL9 and CXCL10 levels generally increase following monotherapy, with an even greater increase following combination treatment. Table 1a presents updated data from Table 1; Table 2a presents updated data from Table 2. Five (5) additional patients were evaluated. Baselines in Tables 1 and 2 represent measurements taken on Day 1. Baselines in Tables 1a and 2a represent an average of two measurements taken at screening and on Day 1. These results are further summarized in FIG. 7 and FIG. 8.

TABLE 1
Patient Serum CXCL9 Levels (pg/mL)

| | | | Post-X4P-001/ |
|---|---|---|---|
| | Baseline | Post-X4P-001 | Pembro |
| Patient | Levels | Monotherapy | Combination |
| 1 | 694 | 794 | 8730 |
| 2 | 1070 | 1380 | 5440 |
| 3 | 556 | 425 | 1930 |
| 4 | 723 | 836 | 4900 |
| 5 | 2980 | 2260 | 12600 |
| 6 | 622 | 2530 | 255 |
| 7 | 790 | 843 | 1390 |
| 8 | 2010 | 2020 | 3830 |
| 9 | 716 | 1020 | 2810 |
| 10 | 1080 | 1410 | 7140 |
| 11 | 355 | 766 | 1310 |

TABLE 1a
Patient Serum CXCL9 Levels (pg/mL)

| | | | Post-X4P-001/ |
|---|---|---|---|
| | Baseline | Post-X4P-001 | Pembro |
| Patient | Levels | Monotherapy | Combination |
| 1 | 694 | 794 | 8730 |
| 2 | 1070 | 1380 | 5440 |
| 3 | 556 | 425 | 1930 |
| 4 | 723 | 836 | 4900 |
| 5 | 2980 | 2260 | 12600 |
| 6 | 622 | 2530 | 255 |
| 7 | 790 | 843 | 1390 |
| 8 | 2010 | 2020 | 3830 |
| 9 | 716 | 1020 | 2810 |
| 10 | 1080 | 1410 | 7140 |
| 11 | 355 | 766 | 1310 |
| 12 | 1245 | 1245 | 8600 |
| 13 | 769.5 | 769.5 | 3090 |
| 14 | 460.5 | 460.5 | 2200 |
| 15 | 1030 | 1030 | 4390 |
| 16 | 1750 | 1750 | 13600 |

TABLE 2
Patient Serum CXCL10 Levels (pg/mL)

| | | | Post-X4P-001/ |
|---|---|---|---|
| | Baseline | Post-X4P-001 | Pembro |
| Patient | Levels | Monotherapy | Combination |
| 1 | 176 | 139 | 573 |
| 2 | 277 | 331 | 942 |
| 3 | 461 | 521 | 1320 |
| 4 | 190 | 767 | 95 |
| 5 | 151 | 146 | 107 |
| 6 | 197 | 262 | 1200 |
| 7 | 59 | 101 | 167 |
| 8 | 131 | 123 | 391 |
| 9 | 331 | 384 | 385 |
| 10 | 107 | 118 | 141 |
| 11 | 186 | 174 | 431 |

TABLE 2a
Patient Serum CXCL10 Levels (pg/mL)

| | | | Post-X4P-001/ |
|---|---|---|---|
| | Baseline | Post-X4P-001 | Pembro |
| Patient | Levels | Monotherapy | Combination |
| 1 | 155 | 139 | 573 |
| 2 | 273.5 | 331 | 942 |
| 3 | 387.5 | 521 | 1320 |
| 4 | 190 | 767 | 95 |
| 5 | 154.5 | 146 | 107 |
| 6 | 261 | 262 | 1200 |
| 7 | 61.5 | 101 | 167 |
| 8 | 124.5 | 123 | 391 |
| 9 | 345.5 | 384 | 385 |
| 10 | 83 | 118 | 141 |
| 11 | 169.5 | 174 | 431 |
| 12 | 130 | 146 | 1110 |
| 13 | 290.5 | 272 | 664 |
| 14 | 146 | 166 | 286 |
| 15 | 251 | 350 | 578 |
| 16 | 212 | | 764 |

Table 3 shows serum biomarker changes compared to baseline at week 4 of treatment with X4P-001 monotherapy.

TABLE 3
Serum Cytokine and Chemokine Biomarker Changes Compared
to Baseline at Week 4 of X4P-001 Monotherapy

| | Signed | Student's |
|---|---|---|
| Biomarker Increase | Rank | t |
| TNF-Related Apoptosis-Inducing Ligand | <0.0001 | <0.0001 |
| Receptor (TRAIL-R3) | | |
| Interleukin-6 Receptor | 0.0002 | 0.0007 |
| (IL-6r) | | |
| Myloid Progenitor Inhibitor Factor | 0.0002 | <0.0001 |
| (MPIF-1) | | |
| Tumor Necrosis Factor Receptor 2 | 0.0004 | 0.0005 |
| (TNFR2) | | |
| Interleukin-2 Simoa | 0.0006 | 0.0063 |
| (IL-2 Simoa) | | |
| Monokine Induced by Gamma Interferon | 0.0012 | 0.0194 |
| (MIG; CXCL9) | | |
| EN-RAGE | 0.0020 | 0.0041 |
| Tumor Necrosis Factor Receptor 1 | 0.0021 | 0.0032 |
| (TNF R1) | | |
| Eotaxin-2 | 0.0026 | 0.1533 |
| Chemokine CC-4 | 0.0034 | 0.0006 |
| (HCC-4) | | |
| Urokinase-type Plasminogen Activator | 0.0034 | 0.0029 |
| Receptor (uPAR) | | |
| Interleukin-2 Receptor Alpha | 0.0103 | 0.0073 |
| (IL-2 receptor alpha) | | |
| Macrophage Inflammatory Protein-1 beta | 0.0103 | 0.0264 |
| (MIP-1 beta) | | |
| Interferon gamma Induced Protein 10 | 0.0157 | 0.1099 |
| (IP-10; CXCL10) | | |
| 6Ckine | 0.0210 | 0.1296 |
| Macrophage Inflammatory Protein 3 beta | 0.353 | 0.0807 |
| (MIP-3 beta) | | |
| Macrophage-Derived Chemokine | 0.353 | 0.0680 |
| (MDC) | | |
| AXL Receptor Tyrosine Kinase | 0.463 | 0.0555 |
| (AXL) | | |
| Tissue Inhibitor of Metalloproteinases 1 | 0.0616 | 0.0278 |
| (TIMP-1) | | |
| Biomarker Decrease | | |
| Plasminogen Activator Inhibitor 1 | 0.0007 | 0.0007 |
| (PAI-1) | | |
| Brain-Derived Neurotrophic Factor | 0.0081 | 0.0040 |
| (BDNF) | | |
| Epidermal Growth Factor | 0.0237 | 0.0302 |
| (EGF) | | |
| E-Selectin | 0.0327 | 0.3136 |
| Monocyte Chemotactic Protein 2 | 0.0377 | 0.0289 |
| (MCP-2) | | |

### Example 3: Serum Biomarkers in Treatment of RCC Patients with Combination of X4P-001 and Nivolumab

Treatment with X4P-001 as a monotherapy, or in combination with a checkpoint inhibitor, such as nivolumab, may be performed in cycles, such as on a 2 week, 4 week, 6 week or 8 week cycle. In certain embodiments, the cycle is 4 weeks long. X4P-001 at a determined dose from 200 mg to 1200 mg daily is administered orally either once daily or twice daily in divided doses. Patients are instructed about both dosing schedule and requirements relating to food or drink near the time of dosing.

Dosing Schedule. The daily dose is taken first thing in the morning. Where the dose is divided, the first daily dose is taken in the morning and the second daily dose approximately 12 hours later using the following guidelines:
Dosing should be at the same time(s) each day±2 hr.
For twice daily dosing, the interval between successive doses should not be <9 hours nor >15 hours. If the interval would be >15 hrs, the dose should be omitted and the usual schedule resumed at the next dose.
Restrictions relating to food. Absorption is impacted by food and patients will be instructed as follows:
For the morning dose
   No food or drink (except water) after midnight until the time of dosing
   No food or drink (except water) for 2 hour after dosing.
For the second daily dose, if applicable
   No food or drink (except water) for 1 hour before dosing
   No food or drink (except water) for 2 hours after dosing.

Nivolumab is administered consistent with prescribed labeling information. Concomitant treatment with X4P-001 and nivolumab may be administered, beginning with daily administration of X4P-001 at day 1. Initial treatment with nivolumab is at 3 mg/kg administered by intravenous infusion over 60 minutes in clinic at the week 4 and 7 visits. Patients may, with the approval of their clinician, vary the dosing schedule or dosage of nivolumab.

Dosing of X4P-001 and/or nivolumab may be adjusted by the clinician as appropriate. The dose of X4P-001 and/or nivolumab may be lowered according to the judgment of the clinician. If a patient receiving X4P-001 in combination with nivolumab experiences an adverse event at Grade>2, the dose of X4P-001 and/or nivolumab may be lowered according to the judgment of the clinician. If a patient successfully completes the first 4 weeks of treatment, that is, without experiencing any adverse events greater than Grade 2, the daily dose of X4P-001 and/or nivolumab may be increased, consistent with the judgment of the clinician.

Evaluation of Response to Treatment and Disease Status. Classification of tumor response may be performed according to codified tumor response evaluation, according to the Response Evaluation Criteria in Solid Tumors Group (“RECIST”), as described in Therasse et al. (2000), J. National Cancer Institute, 92:205-216. Radiologic assessment of ccRCC is accomplished by Computed Tomography (CT) with slice thickness≤5 mm and contrast. CT is performed prior to treatment (baseline) and may be made at intervals during treatment to determine the response.

### KEY TERMINOLOGY

Measurable non-nodal lesions—≥10 mm in longest diameter.

Measurable nodal lesions—≥15 mm in short axis

Nonmeasurable lesions—lesions that are smaller, including those that cannot be measured.

Measurable disease—presence of at least one measurable lesion.

### Target Lesions

At baseline, four (4) measurable lesions, two (2) for each individual organ, are identified, documented, and the appropriate diameter of each is recorded. If measurable extra-renal lesions are present, a measurable extra-renal lesion is also identified, documented, and the appropriate diameter is recorded. Lesions are selected based on size, to be representative of disease, and suitable for reproducible repeat measurement. Target lesions may include measurable lymph nodes.

During treatment, each target lesion is assessed for Complete Response, Partial Response, Stable Disease, or Progressive Disease as follows:

Complete Response (CR)
(a) Disappearance of all non-nodal lesions, and
(b) Absence of pathologic lymph nodes^{a}.

Partial Response (PR)
(a) ≥30% decrease from baseline in the SOD of the target lesions Stable Disease (SD)
(a) Persisting disease that does not meet criteria for either PR or PD Progressive Disease (PD)
(a) ≥20% increase in the SOD of the target lesions, compared to the smallest sum, which may be either at baseline or while on treatment; and
(b) an absolute increase of ≥5 mm in the SOD.

### Non-Target Lesions

All other lesions present at baseline, including pathologic nodes (defined as nodes>10 mm in short axis) should be documented (quantitative measurements are not required) so that they can be classified on follow-up as present, absent, or unequivocal progression.

Complete Response (CR)
(a) Disappearance of all non-target lesions, and
(b) Absence of pathologic lymph nodes^{a}.

Non-CR/non-PD
Persistence of one or more non-target lesions

Progressive Disease (PD)
Unequivocal progression of existing non-target lesions.
   [Note: a=All lymph nodes, whether or not designated target or non-target lesions, have short axis diameter≤10 mm]

### New Lesions

A new lesion should be unequivocal (e.g., not attributable to variation in technique); includes lesions in a location not scanned at baseline.

### Pharmacokinetic Assessments

If desired, pharmacokinetic assessment of blood samples for plasma levels of X4P-001 and nivolumab may be conducted. Blood samples are collected as scheduled. Samples are analyzed for X4P-001 concentration using reversed-phase high performance liquid chromatography (RP-HPLC) with MS/MS detection. The validated range of this bioanalytic method is 30 to 3,000 ng/mL in plasma.

Pharmacokinetic assessment of nivolumab may be accomplished using techniques, such as those described in Glassman and Balthasar (2014) Cancer Biol. Med. 11:20-33; Wang et al. (2014), Cancer Immunology Research, 2:1-11; or the Assessment Report of the European Medicines Agency (EMA) for nivolumab EMEA, assessment report EMA/CHMP/76688/2015, Apr. 23, 2015. The full disclosure of these documents are hereby specifically incorporated herein by reference.

Following a clinical protocol consistent with the disclosure of WO 2017/177230, the disclosure of which is hereby incorporated herein by reference, total of 9 patients were enrolled in a clinical trial to evaluate the safety and tolerability of X4P-001 in combination with nivolumab in patients who are unresponsive to nivolumab monotherapy. The secondary and exploratory objectives were to characterize the antitumor activity of X4P-001 and nivolumab combination treatment; and investigate select peripheral blood biomarkers of immune activation.

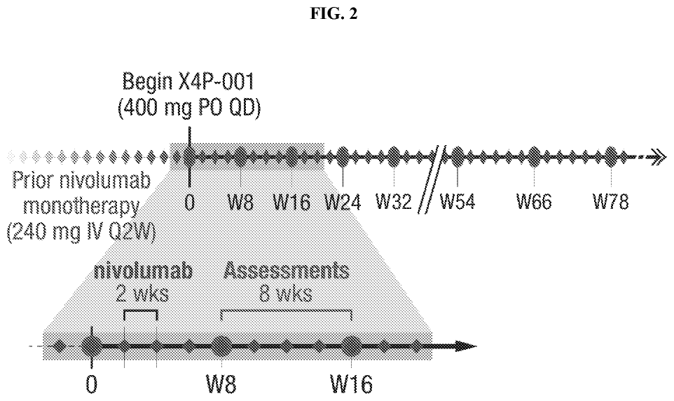

As shown in FIG. 2, the starting dose of X4P-001 was chosen based on safety and pharmacological activity in healthy volunteers and prior RCC studies we conducted. Oral X4P-001 was administered to patients at 400 mg QD while continuing on 240 mg nivolumab therapy by IV infusion every 2 weeks. Radiologic assessments for tumor response were conducted every 8 weeks during the first 12 months and every 12 weeks thereafter, or as warranted based on RECIST v1.1 criteria.

Key eligibility criteria were:
Patient was receiving current nivolumab monotherapy
Best response on current nivolumab of SD or PD
Histologically confirmed RCC with documented clear cell component
≥18 years of age

Key exclusion criteria were:
<3 month life expectancy
ECOG performance status≥2
Screening laboratory tests of ANC<1,500/μL or platelets<75,000/μL
Active CNS metastasis or uncontrolled heart disease

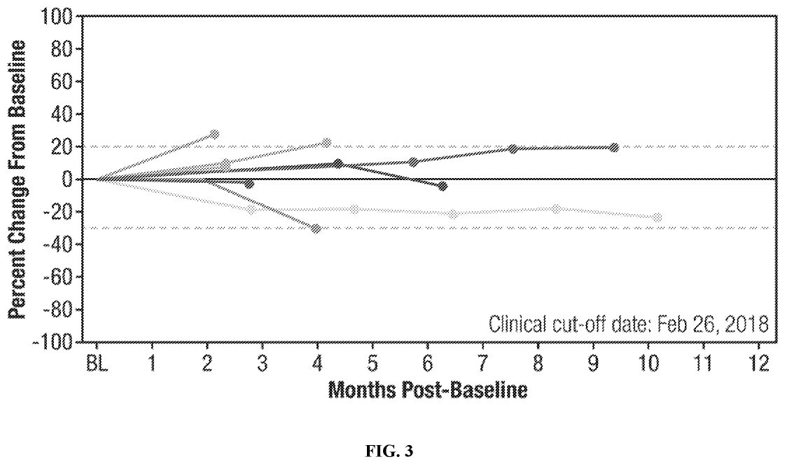

A total of 9 patients were treated, of whom 2 (22%) remained in ongoing treatment after 10 months, and 7 discontinued due to an adverse event (AE) (3, 33%), disease progression (3, 33%), or clinical deterioration (1, 11%). Combination therapy was discontinued in 3 patients for AEs of Lipase Increased, Mucosal Inflammation/Rash Maculo-Papular, and Autoimmune Hepatitis (1 each). The median duration of combination treatment was 3.7 months (range 1-10 months). X4P-001+nivolumab combination therapy had acceptable toxicity in RCC patients. There were no Grade 4 or Grade 5 AEs. FIG. 3 shows the target lesion response over time.

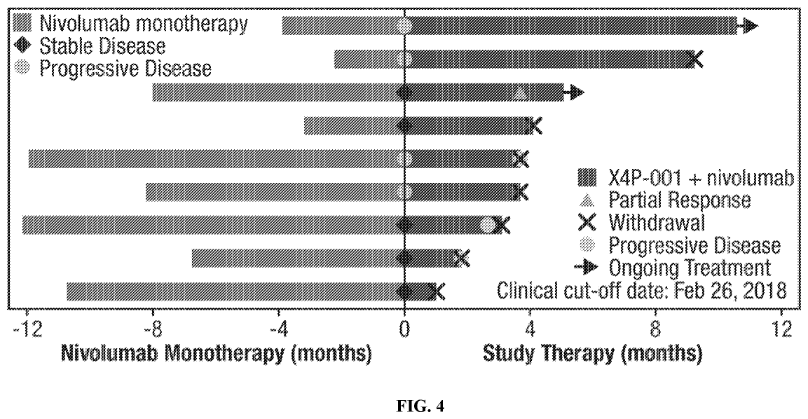

FIG. 4 shows the duration of prior nivolumab monotherapy and combination treatment and patient responses. Four patients with progressive disease on prior nivolumab monotherapy had a best response of stable disease (SD) with X4P-001+nivolumab. Among 5 patients with stable disease on prior nivolumab monotherapy, 1 had a partial response (PR) with X4P-001+nivolumab.

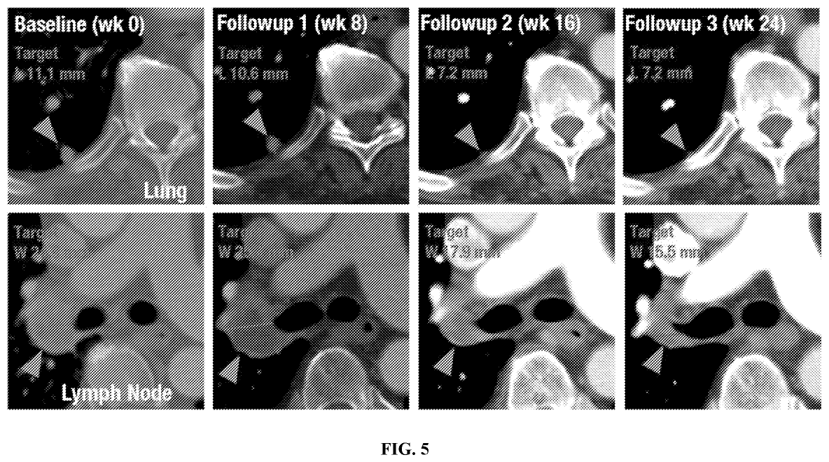

FIG. 5 shows an assessment of tumor responses by CT scans for a patient receiving X4P-001+nivolumab combination therapy that had a partial response. Top row: Target lesion in the lung. Bottom row: lymph node target lesion. Scans were taken every 8 weeks and target lesion size was determined per RECIST v1.1 criteria.

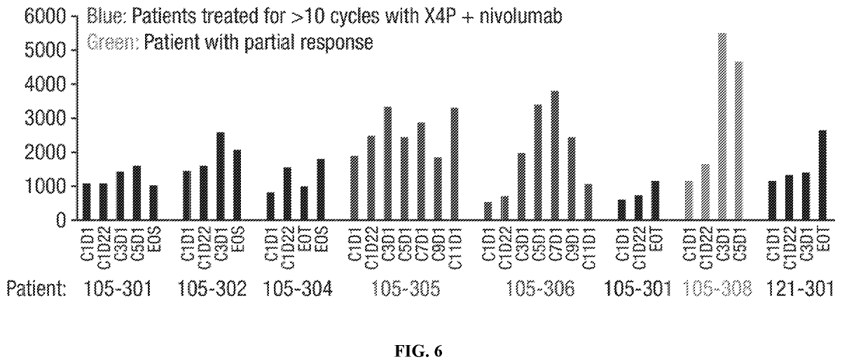

FIG. 6 shows measured increases in CXCL9 (MIG) levels in patients treated with X4P-001+Nivolumab. Higher CXCL9 levels were found in a patient with a partial response (PR) and in those receiving combination therapy for >10 cycles. CXCL9 levels shown in FIG. 6 are also presented below in Table 4.

TABLE 4

| | | Monokine |
|---|---|---|
| | | Induced by |
| | | Gamma |
| | | Interferon |
| | | (MIG) |
| 105-301 | C1D1 | 1090 |
| 105-301 | C1D22 | 1080 |
| 105-301 | C3D1 | 1440 |
| 105-301 | C5D1 | 1600 |
| 105-301 | EOS | 1020 |
| 105-302 | C1D1 | 1450 |
| 105-302 | C1D22 | 1600 |
| 105-302 | C3D1 | 2580 |
| 105-302 | EOS | 2070 |
| 105-304 | C1D1 | 807 |
| 105-304 | C1D22 | 1560 |
| 105-304 | EOT | 991 |
| 105-304 | EOS | 1800 |
| 105-305 | C1D1 | 1880 |
| 105-305 | C1D22 | 2490 |
| 105-305 | C3D1 | 3340 |
| 105-305 | C5D1 | 2440 |
| 105-305 | C7D1 | 2870 |
| 105-305 | C9D1 | 1840 |
| 105-305 | C11D1 | 3320 |
| 105-306 | C1D1 | 542 |
| 105-306 | C1D22 | 712 |
| 105-306 | C3D1 | 1970 |
| 105-306 | C5D1 | 3390 |
| 105-306 | C7D1 | 3810 |
| 105-306 | C9D1 | 2440 |
| 105-306 | C11D1 | 1060 |
| 105-307 | C1D1 | 603 |
| 105-307 | C1D22 | 722 |
| 105-307 | EOT | 1160 |
| 105-308 | C1D1 | 1160 |
| 105-308 | C1D22 | 1630 |
| 105-308 | C3D1 | 5500 |
| 105-308 | C5D1 | 4660 |
| 121-301 | C1D1 | 1170 |
| 121-301 | C1D22 | 1320 |
| 121-301 | C3D1 | 1420 |
| 121-301 | EOT | 2650 |
| 121-301 | EOS | 925 |

Serum sample collection time points in renal cell carcinoma (RCCB) were:
C1D1 (pre-dose), C1D22, C3D1, C5D1, C7D1 . . . EOT, EOS. C1=Cycle 1, C3=Cycle 3, etc.; D1=Dose 1, D22=Dose 22, etc.; EOT=End of Treatment; EOS=End of Study.

A total of 93 chemokines/cytokines/growth factors were measured. 12 were below the detection limit.

Among the 81 proteins:
24 changed with p<0.05 at at least one time point relative to the baseline value at C1D1.
Most changes took place at C1D22 and C3D1.

The Myriad-RBM MAP Platform was used to analyze biomarkers.

Table 5 shows the best overall response and objective response rate.

TABLE 5
Best Overall Response X4P-001 + Nivolumab (n = 9)

| Best Overall Response* | |
|---|---|
| Partial Response (PR) | 1 (11%) |
| Stable Disease (SD) | 7 (78%) |
| Progressive Disease (PD) | 1 (11%) |
| Objective Response Rate (CR + PR) | 11% |

*Based upon RECIST 1.1.

Table 6 shows serum biomarker changes compared to baseline on Day 22 of treatment with X4P-001+nivolumab combination therapy.

TABLE 6
Serum Biomarker Changes Compared to Baseline on Day 22 of X4P-
001 + Nivolumab Combination Therapy (p < 0.05)

| Protein* | p-value |
|---|---|
| 6Ckine (increased) | 0.016 |
| Angiopoietin-1 (ANG-1) (decreased) | 0.031 |
| Decorin (increased) | 0.008 |
| Epithelial-Derived Neutrophil-Activating | 0.031 |
| Protein 78 (ENA-78) (decreased) | |
| Interleukin-2 Simoa (IL-2 Simoa) (increased) | 0.023 |
| Latency-Associated Peptide of Transforming | 0.016 |
| Growth Factor beta 1 (decreased) | |
| Macrophage inflammatory protein 3 beta | 0.016 |
| (MIP-3 beta) (increased) | |
| Monocyte Chemotactic Protein 1 (MCP-1) | 0.016 |
| CXCL9, Monokine Induced by Gamma | 0.016 |
| Interferon (MIG) (increased) | |
| Myeloid Progenitor Inhibitory Factor 1 | 0.031 |
| (MPIF-1) (increased) | |
| Platelet-Derived Growth Factor BB (PDGF- | 0.023 |
| BB) (decreased) | |

*Determined using the Multi-Analyte Profile platform (Myriad RBM).

In conclusion, combination therapy with X4P-001 (400 mg QD)+nivolumab exhibited some anti-tumor activity and was tolerable in advanced RCC patients that were previously unresponsive to nivolumab monotherapy. CXCR4 inhibition by X4P-001 can potentially augment responses in patients that do not respond to anti-PD-1 checkpoint inhibitors alone. Serum biomarker analyses identified significant early changes in cytokines and chemokines, including CXCL9, a chemoattractant ligand for cytotoxic T cell migration.

The entire disclosure of each of the patent documents and scientific articles cited herein is incorporated by reference for all purposes.

The invention can be embodied in other specific forms without departing from the essential characteristics thereof. The foregoing embodiments therefore are to be considered illustrative rather than limiting on the invention described herein. Modifications and alternative embodiments will be readily apparent to the skilled artisan upon reading the specification, claims and accompanying drawings and figures. The scope of the invention is indicated by the appended claims rather than by the foregoing description, and all changes that come within the meaning and range of equivalency of the claims are intended to be embraced therein.

We claim:

1. A method of identifying a patient with a cancer who will benefit from treatment with a CXCR4 inhibitor optionally in combination with an immunotherapeutic agent, comprising:
(a) obtaining a first serum sample prior to administration of the CXCR4 inhibitor to the patient;
(b) measuring a level in the first serum sample of one or more biomarkers selected from a cytokine panel, a cytokine signature, a ratio of one or more cytokine ratios, or a cytokine score;
(c) administering to the patient an effective amount of a CXCR4 inhibitor and optionally an immunotherapeutic agent;
(d) obtaining a second serum sample after administration of the CXCR4 inhibitor to the patient; and
(e) measuring a level in the second serum sample of one or more biomarkers selected from a cytokine panel, a cytokine signature, a ratio of one or more cytokine ratios, or a cytokine score;
wherein the cancer response to step (c) is predictive of the likelihood of successful treatment of the cancer based on a greater or lesser response of the cancer compared with one or more similar patients and as evaluated using one or more of the biomarkers.

2. A method of predicting a cancer patient's response to a CXCR4 inhibitor optionally in combination with an immunotherapeutic agent in a patient, comprising the steps of:
(a) obtaining a first serum sample prior to administration of the CXCR4 inhibitor to the patient;
(b) measuring a level in the first serum sample of one or more biomarkers selected from a cytokine panel, a cytokine signature, a ratio of one or more cytokine ratios, or a cytokine score;
(c) administering to the patient an effective amount of a CXCR4 inhibitor and optionally an immunotherapeutic agent;
(d) obtaining a second serum sample after administration of the CXCR4 inhibitor to the patient; and
(e) measuring a level in the second serum sample of one or more biomarkers selected from a cytokine panel, a cytokine signature, a ratio of one or more cytokine ratios, or a cytokine score;
wherein the cancer response to step (c) is predictive of the likelihood of successful treatment of the cancer based on a greater or lesser response of the cancer compared with one or more similar patients and as evaluated using one or more of the biomarkers.

3. The method of claim 1 or 2, wherein the CXCR4 inhibitor is X4P-001 or a pharmaceutically acceptable salt thereof.

4. The method of any one of claims 1-3, wherein the immunotherapeutic agent is an immune checkpoint inhibitor.

5. The method of claim 4, wherein the immune checkpoint inhibitor is selected from ipilimumab (Yervoy®), atezolizumab (Tecentriq®), nivolumab (Opdivo®), pidilizumab, avelumab (Bavencio®), durvalumab (Imfinzi®), PDR001, REGN2810, or pembrolizumab (Keytruda®).

6. The method of claim 5, wherein the immune checkpoint inhibitor is pembrolizumab or nivolumab.

7. The method of any one of claims 1-6, wherein the cytokine panel comprises an increase in one or more of IL-6, IL-7, IL-8, IL-10, IL-12p70, IL-22, IL-23p19, IFN-α2, IFN-γ, TNF-β, MCP-1, SDF-1, CXCL10, CXCL9, GM-CSF, PDGF, HGF, and VEGF-A.

8. The method of claim 7, wherein the cytokine panel comprises an increase in one or more of IFN-γ, CXCL10, and CXCL9.

9. The method of claim 7, wherein the cytokine panel comprises an increase in CXCL10 or CXCL9.

10. The method of any one of claims 1-9, wherein the cancer is selected from renal cell cancer, melanoma, liver cancer, hepatocellular carcinoma, hepatocholangiocarcinoma, ovarian cancer, ovarian epithelial cancer, fallopian tube cancer, papillary serous cystadenocarcinoma, uterine papillary serous carcinoma (UPSC); prostate cancer; testicular cancer, gall bladder cancer, adrenocortical adenocarcinoma, colon cancer, pancreatic cancer, pancreatic carcinoma, brain cancer, gastrointestinal/stomach (GIST) cancer, medulloblastoma, glioma, glioblastoma, squamous cell carcinoma of the head and neck (SCCHN), Waldenstrom's macroglobulinemia, breast cancer, urothelial carcinoma, head and neck cancer, or cervical cancer.

11. The method of claim 10, wherein the cancer is advanced or metastatic melanoma.

12. The method of claim 10 or 11, wherein the melanoma is unresectable advanced or unresectable metastatic melanoma.

13. The method of any one of claims 1-9, wherein the cancer is renal cell carcinoma (RCC).

14. The method of any one of claims 1-6, wherein the biomarker is a cytokine panel.

15. The method of claim 14, wherein the cytokine panel comprises one or more biomarkers selected from ANG-1, ENA-78, Latency-associated peptide of transforming growth factor beta 1, MCP-1, and PDGF-BB.

16. The method of claim 14 or 15, wherein the cytokine panel comprises one or more biomarkers selected from 6CKine, Decorin, IL-2, MIP-3 beta, MIG (CXCL9), and MPIF-1P.

17. The method of claim 16, wherein the expression level of one or more of ANG-1, ENA-78, Latency-associated peptide of transforming growth factor beta 1, MCP-1, and PDGF-BB is decreased after administration of the CXCR4 inhibitor.

18. The method of any one of claims 14-17, wherein the expression level of one or more of 6CKine, Decorin, IL-2, MIP-3 beta, MIG (CXCL9), and MPIF-1P is increased after administration of the CXCR4 inhibitor.

19. The method of claim 18, wherein the expression level of one or more of ANG-1, ENA-78, Latency-associated peptide of transforming growth factor beta 1, MCP-1, and PDGF-BB is decreased after administration of the CXCR4 inhibitor and the expression level of one or more of 6CKine, Decorin, IL-2, MIP-3 beta, MIG (CXCL9), and MPIF-1P is increased after administration of the CXCR4 inhibitor.

20. The method of claim 18, wherein the expression level of each of ANG-1, ENA-78, Latency-associated peptide of transforming growth factor beta 1, MCP-1, and PDGF-BB is decreased after administration of the CXCR4 inhibitor and the expression level of each of 6CKine, Decorin, IL-2, MIP-3 beta, MIG (CXCL9), and MPIF-1P is increased after administration of the CXCR4 inhibitor.

21. The method of any one of claims 14-20, wherein the cancer is selected from renal cell cancer, melanoma, liver cancer, hepatocellular carcinoma, hepatocholangiocarcinoma, ovarian cancer, ovarian epithelial cancer, fallopian tube cancer, papillary serous cystadenocarcinoma, uterine papillary serous carcinoma (UPSC); prostate cancer; testicular cancer, gall bladder cancer, adrenocortical adenocarcinoma, colon cancer, pancreatic cancer, pancreatic carcinoma, brain cancer, gastrointestinal/stomach (GIST) cancer, medulloblastoma, glioma, glioblastoma, squamous cell carcinoma of the head and neck (SCCHN), Waldenstrom's macroglobulinemia, breast cancer, urothelial carcinoma, head and neck cancer, or cervical cancer.

22. The method of claim 21, wherein the cancer is advanced or metastatic melanoma.

23. The method of claim 21 or 22, wherein the melanoma is unresectable advanced or unresectable metastatic melanoma.

24. The method of claim 21, wherein the cancer is renal cell carcinoma (RCC).

25. The method of any one of claims 1-6, wherein the cytokine panel comprises an increase in one or more of TRAIL-R3, IL-6r, MPIF-1, TNFR2, IL-2 Simoa, CXCL9, EN-RAGE, TNF R1, Eotaxin-2, HCC-4, uPAR, IL-2 receptor alpha, MIP-1 beta, CXCL10, 6Ckine, MIP-3 beta, MDC, AXL, and TIMP-1.

26. The method of any one of claims 1-6, wherein the cytokine panel comprises a decrease in one or more of PAI-1, BDNF, EGF, E-Selectin, and MCP-2.

27. The method of claim 14, wherein the cytokine panel comprises one or more biomarkers selected from TRAIL-R3, IL-6r, MPIF-1, TNFR2, IL-2 Simoa, CXCL9, EN-RAGE, TNF R1, Eotaxin-2, HCC-4, uPAR, IL-2 receptor alpha, MIP-1 beta, CXCL10, 6Ckine, MIP-3 beta, MDC, AXL, TIMP-1, PAI-1, BDNF, EGF, E-Selectin, and MCP-2.

28. The method of claim 27, wherein the expression level of one or more of TRAIL-R3, IL-6r, MPIF-1, TNFR2, IL-2 Simoa, CXCL9, EN-RAGE, TNF R1, Eotaxin-2, HCC-4, uPAR, IL-2 receptor alpha, MIP-1 beta, CXCL10, 6Ckine, MIP-3 beta, MDC, AXL, and TIMP-1 is increased after administration of the CXCR4 inhibitor.

29. The method of claim 27, wherein the expression level of one or more of PAI-1, BDNF, EGF, E-Selectin, and MCP-2 is decreased after administration of the CXCR4 inhibitor.

30. The method of claim 9, wherein the biomarker comprises a change in the serum concentration of CXCL9 and/or CXCL10 in a patient after 1, 2, 3, 4, 5, 6, 7, 8, 9, or more weeks of treatment.

31. The method of claim 30, wherein the change in the serum concentration of CXCL9 is increased after treatment by at least about 4.5-fold.

32. The method of claim 30, wherein the change in the serum concentration of CXCL10 is increased after treatment by at least about 2.5-fold.

33. A method of predicting a treatment response of a cancer in a patient to an immunotherapeutic agent in combination with a CXCR4 inhibitor, comprising the steps of:
(a) obtaining a first serum sample from a patent prior to the administration of the CXCR4 inhibitor to the patient;
(b) measuring a level in the first serum sample of one or more biomarkers selected from a cytokine panel, cytokine signature, a ratio of one or more cytokine ratios, or a cytokine score;
(c) administering to the patient an effective amount of a CXCR4 inhibitor and optionally an immunotherapeutic agent;
(d) obtaining a second serum sample after administration of the CXCR4 inhibitor to the patient;
(e) measuring a level in the second serum sample of one or more biomarkers selected from a cytokine panel, cytokine signature, a ratio of one or more cytokine ratios, or a cytokine score;
wherein the tumor response to step (c) is predictive of the likelihood of successful treatment of the tumor with an immunotherapeutic agent after treatment with a CXCR4 inhibitor, based on a greater of lesser response of the tumor compared with one or more similar patients and as evaluated using one or more biomarkers.

34. The method of claim 33, wherein the CXCR4 inhibitor is X4P-001.

35. The method of claim 33 or 34, wherein the immunotherapeutic agent is a checkpoint inhibitor.

36. The method of claim 35, wherein the patient initially does not respond to treatment to the checkpoint inhibitor.

37. The method of claim 35, wherein the patient initially responded to treatment with a checkpoint inhibitor, but has become refractory to treatment with the checkpoint inhibitor.

38. The method of claim 36 or 37, wherein the biomarker is selected from CXCL9 or CXCL10.
